(19) United States
(12) Patent Application Publication
(10) Pub. No.: US 2005/0059014 A1
(43) Pub. Date: Mar. 17, 2005

(54) Analytical platform and detection method with the analytes to be determined in a sample as immobilized specific binding partners

(76) Inventors: Michael Pawlak, Laufenburg (DE); Eginhard Schick, Rheinfelden (DE); Peter Oroszlan, Basel (CH)

Correspondence Address: WENDEROTH, LIND & PONACK, L.L.P., 2033 K STREET N. W., SUITE 800, WASHINGTON, DC, 20006-1021, US

(21) Appl. No.: 10/653,386

(22) Filed: Sep. 3, 2003

(30) Foreign Application Priority Data
Sep. 2, 2002 (CH) 1502/02
Jan. 27, 2003 (CH) 0114/03

(51) Int. Cl. C12Q 1/68

(52) U.S. Cl. 435/6

(57) ABSTRACT

The present invention is related to an analytical platform and a method performed therewith for the analysis of a multitude of “nature-identical” samples for analytes which are contained therein and are of biological relevance as binding partners in specific binding reactions, wherein said samples or dilutions of said samples of the same relative molecular composition as the original samples, with the analytes to be determined contained therein, as a first plurality of specific binding partners, are deposited in discrete measurement areas in at least one one- or two-dimensional array of measurement areas on an evanescent field sensor platform as a solid support without a change in the relative molecular composition, compared with the original relative molecular composition of the sample, one or more tracer compounds as a second plurality of specific binding partners, for the specific determination of analytes out of the first plurality of specific binding partners contained in the samples, are brought into contact with the samples deposited in said discrete measurement areas in a single step or multiple steps of a specific binding reaction, changes in opto-electronic signals, resulting from the binding of tracer compounds to analytes contained in the samples in discrete measurement areas or to specific binding partners bound to the analytes contained in the measurement areas in the evanescent field of the evanescent field sensor platform are measured laterally resolved, and the presence of the analytes to be specifically detected is determined qualitatively and/or quantitatively from the relative amount of the changes in said opto-electronic signals from the corresponding measurement areas.

[symbol] platform and a method performed [symbol] ture-identical” samples for analytes [symbol] e as binding partners in specific binding [symbol] es of the same relative molecular [symbol] th the analytes to be determined [symbol] specific binding partners, are deposited [symbol] st one one- or two-dimensional array of [symbol] eld sensor platform as a solid support [symbol] lar composition, compared with the [symbol] of the sample, [symbol] ond plurality of specific binding [symbol] of one or more analytes from the first [symbol] ntained in the samples, are brought into [symbol] aid discrete measurement areas in a [symbol] ic binding reaction, [symbol] lting from the binding of tracer [symbol] samples in discrete measurement areas [symbol] t field sensor platform are measured [symbol] etected is determined qualitatively and [symbol] of the changes in said opto-electronic [symbol] reas. [symbol] s, resulting from the binding of tracer [symbol] s in discrete measurement areas in the [symbol] determined, for example, from the [symbol] gnals from different measurement areas [symbol] wn or unknown concentration and/or [symbol] eas which do not contain the [symbol] he determination of said signal changes, also the signals from measurement areas with unknown concentrations of analytes and the signals from measurement areas containing analytes at a known concentration may be used. In the case of a continuous signal acquisition during and after application of the corresponding tracer compounds and their binding to the corresponding analytes contained in the measurement areas, a corresponding signal change can also be determined from the temporal evolution of the signals from the corresponding measurement areas.

In the following (and particularly with regard to the claims of the present patent application) the term “a” (“nature-identical”) sample is always also related to two or more, i.e. multiple (“nature-identical”) samples, unless explicitly stated otherwise.

For many fields of application, multiple biologically relevant analytes need to be determined in a complex sample, for example, in diagnostic methods for determining an individuals state of the health or in pharmaceutical research or development for determining the effects of the administration of biologically active compounds on an organism and on its complex functional mode.

Whereas known analytical separation methods have in general been optimized to separate the largest possible number of compounds contained in a given sample within the shortest possible time, according to a given physical-chemical parameter, such as the molecular weight or the ratio of the molecular charge and the mass, bioaffinity-related methods of determination are based on recognizing and binding with high selectivity the corresponding (single) analyte of interest in a sample of complex content by a biological or biochemical or synthetic recognition element the greatest possible specificity. The determination of many different compounds thus requires the application of a correspondingly large number of different specific recognition elements.

A determination method based on a bioaffinity reaction can be performed both in a homogeneous solution and at the surface of a solid support. Depending on the specific method, washing steps may be required after binding of the analytes to the recognition elements and of optional further tracer compounds and optionally between different steps of the process in order to separate the complexes formed between the recognition elements and the analytes to be determined and optional further tracer compounds from the residual part of the sample and of the additional indicator reagents that are optionally applied.

Methods for the simultaneous determination of many different nucleic acids in a sample using corresponding complementary nucleic acids as recognition elements immobilized in discrete, laterally separated measurement areas on a solid support are in relatively wide use nowadays. For example, arrays of oligonucleotides based on simple glass or microscope plates are known as recognition elements with a very high feature density (density of measurement areas on a common solid support). For example, in U.S. Pat. No. 5,445,934 (Affymax Technologies) arrays of oligonucleotides with a density of more than 1000 features per square centimeter have been described and claimed.

Recently, there have also been frequent descriptions of similar arrays and methods based thereon for simultaneous determination of multiple proteins, for example in U.S. Pat. No. 6,365,418 B1.

The disclosures for such so-called “microarrays” for the determination both of nucleic acids and of other biopolymers, such as proteins, describe how multiple specific recognition elements are immobilized in discrete measurement areas in order to generate an array for analyte recognition and are then brought into contact with the sample to be analyzed, comprising the analytes, perhaps in a complex mixture. Following the known disclosures, different specific recognition elements are provided in as pure a form as possible in separate discrete measurement areas, so that generally different analytes will bind to measurement areas with different recognition elements.

For this kind of known assay, it is required that the specific recognition elements to be immobilized in as pure a quality as possible be enriched by means of what in some cases are very laborious steps. As different recognition elements also differ more or less in terms of their physical-chemical properties (for example, their polarity), there are also corresponding differences in the conditions for their optimized immobilization in discrete measurement areas on a common support, optionally mediated by an adhesion-promoting layer, for example, by adsorption or by covalent binding. Accordingly, the conditions chosen for immobilizing multiple different recognition elements (such as the nature of the adhesion-promoting layer) can hardly be optimal for all recognition elements to be immobilized, but will generally be a compromise between the immobilization properties of the different recognition elements of interest.

Furthermore, a disadvantage with this kind of assay is that, for the determination of analytes in a certain number of samples, it is necessary to provide a corresponding number of discrete arrays on a common support or on discrete supports to which the different samples are applied. For the analysis of multiple different samples, this implies the need for a large number of discrete arrays, the manufacture of which is relatively complex.

It has been described, for example, that under suitable conditions for dissociation the hybrids formed between immobilized oligonucleotides and complementary oligonucleotides supplied in a sample may be dissociated with high efficiency and a recognition surface thus be “regenerated”; however, a 100% regeneration can hardly be guaranteed. In the case of bioaffinity complexes with proteins, the complexation step is often not even reversible, i.e. the recognition surface cannot be regenerated.

There is therefore a need for a modified assay architecture enabling multiple samples in a single array on a common support to be analyzed for the analytes contained in said samples simultaneously. For this purpose it would be useful to immobilize not the different specific recognition elements, but the samples to be analyzed themselves, if possible directly, without further pre-treatment, or after as low a number of pre- treatment steps as possible, on a support. In the following, an assay architecture of this type shall be called an “inverted assay architecture”.

In U.S. Pat. No. 6,316,267 a method is described, wherein polyamino acids (possibly in a complex sample mixture) are, for example, applied on solid or a “semi-solid” sample matrix. The detection step, however, is performed not in a bioaffinity assay, but by staining using a mixture of reagents comprising certain metal complexes exemplified in said disclosure. This is obviously not a method of specific analyte detection.

In U.S. Pat. No. 6,287,768 a method is described, wherein different RNA molecules to be determined from a biological sample are isolated, separated by size, deposited on a solid support and then determined thereon, for example in a hybridization assay upon hybridization with known, complementary polynucleotides. According to the disclosure in that patent, either the RNA molecules to be determined and isolated from an organism can be subjected directly to the further determination method, if they are present in high abundance, or they have to be amplified beforehand by known amplification methods (e.g. by polymerase chain reaction, “PCR”).

Although the method proposed in U.S. Pat. No. 6,287,768 opens the opportunity to determine RNA from different samples simultaneously, it still requires numerous elaborate sample preparation steps and in particular isolation from the biological sample matrix, followed by a separation of the sample according to molecular size. In view of the fact that the claimed method, which is only described with reference to the example of RNA, requires at least isolation from the original sample matrix and separation of the biopolymers according to size, it has to be expected that the relative molecular composition, after this separation step and before the analysis step, will be different from the relative molecular composition of the original sample.

Here and in the following the attribute of an “unchanged relative molecular composition” shall mean that the ratio of the concentrations of the analytes to be determined in an analysis remains unchanged. Following this nomenclature, changes in the content of solvent or matrix molecules or of other molecules which are not determined in the corresponding determination method will be disregarded when using this attribute.

The fact that the detection steps applied in these methods are generally not sensitive enough for achieving required detection limits for the analytes to be determined in the samples can be seen as a reason for including the described separation or enrichment steps in the named analysis methods.

The excitation of “tracer compounds” (such as radioactive isotopes or chromophores with a characteristic absorption and/or luminescence or fluorescence) applied for analyte detection and the read-out of the signals from arrays as described is based on classical optical arrangements and detection methods. The classical measurement methods, such as measurements of absorption or fluorescence, are based in general on direct illumination of a sample volume in a sample compartment or of a measurement field on the inner wall of a sample compartment of a liquid sample. A disadvantage of such arrangements is that, besides collecting signals from the excitation volume or the excitation area wherein a signal for analyte determination is generated, a significant part of the environment is generally exposed to excitation light, which can lead to the disadvantageous generation of disturbing background signals.

For achieving lower detection limits, numerous measurement arrangements have been developed wherein the determination of an analyte is based on its interaction with the evanescent field which is associated with light guiding in an optical waveguide.

When a light wave is coupled into an optical waveguide surrounded by optically rarer media, i.e., media of lower refractive index, the light wave is guided by total reflection at the interfaces of the waveguiding layer. In that arrangement, a fraction of the electromagnetic energy penetrates the media of lower refractive index. This portion is termed the evanescent (=decaying) field. The strength of the evanescent field depends to a very great extent on the thickness of the waveguiding layer itself and on the ratio of the refractive indices of the waveguiding layer and of the media surrounding it. In the case of thin waveguides, i.e. waveguides with layer thicknesses that are the same as or smaller than the wavelength of the light to be guided, discrete modes of the guided light can be distinguished. Such methods have the advantage that the interaction with the analyte is limited to the penetration depth of the evanescent field into the adjacent medium, being of the order of some hundred nanometers, and interfering signals from the depth of the (bulk) medium can be largely avoided. The first proposed measurement arrangements of this type were based on highly multi-modal, self-supporting single layer waveguides, such as fibers or plates of transparent plastic or glass, with thicknesses from some hundred micrometers up to several millimeters.

To improve sensitivity and at the same time simplify manufacture, planar thin-film waveguides have been proposed. In the simplest case, a planar thin-film waveguide consists of a three-layer system: support material (substrate), waveguiding layer, superstrate (the sample to be analyzed), wherein the waveguiding layer has the highest refractive index.

Several methods for the incoupling of excitation light into a planar waveguide are known. The earliest methods used were based on butt coupling or prism coupling, wherein generally a liquid is introduced between the prism and the waveguide, in order to reduce reflections resulting from air gaps. These two methods are suitable in particular with waveguides of relatively large layer thickness, i.e. especially self-supporting waveguides, and with waveguides whose refractive index is substantially less than 2. For incoupling of excitation light into very thin waveguiding layers with a high refractive index, however, the use of coupling gratings is a significantly more elegant method.

Different methods of analyte determination in the evanescent field of lightwaves guided in optical film waveguides can be distinguished. According to the measurement principle used, for example, a distinction can be drawn between fluorescence, or more general luminescence methods on the one hand and refractive methods on the other. In this context, methods for generating surface plasmon resonance in a thin metal layer on a dielectric layer of lower refractive index can be included in the group of refractive methods, if the resonance angle of the launched excitation light for generating the surface plasmon resonance is taken as the quantity to be measured. Surface plasmon resonance can also be used for amplifying a luminescence or for improving the signal-to-background ratio in a luminescence measurement. The conditions for generating a surface plasmon resonance and for combining it with luminescence measurements, as well as with waveguiding structures, are described in the literature, for example in U.S. Pat. No. 5,478,755, U.S. Pat. No. 5,841,143, U.S. Pat. No. 5,006,716, and U.S. Pat. No. 4,649,280.

In this application, the term “luminescence” means the spontaneous emission of photons in the range from ultraviolet to infrared, after optical or nonoptical excitation, such as electrical or chemical or biochemical or thermal excitation. For example, chemiluminescence, bioluminescence, electroluminescence, and especially fluorescence and phosphorescence are included under the term “luminescence”.

In the case of refractive measurement methods, the change in the so-called effective refractive index resulting from molecular adsorption to or desorption from the waveguide is used for analyte detection. This change in the effective refractive index is determined, in the case of grating coupler sensors, from changes in the coupling angle for the in- or out-coupling of light into or out of the grating coupler sensor and, in the case of interferometric sensors, from changes in the phase difference between measurement light guided in a sensing arm and a reference arm of the interferometer.

The aforesaid refractive methods have the advantage that they can be applied without using additional marker molecules, so-called molecular labels. The disadvantage of these label-free methods, however, is that—because of the lower selectivity of the measurement principle—the detection limits which can be achieved with these methods are limited to pico- to nanomolar concentration ranges, depending on the molecular weight of the analyte, and this is not sufficient for many applications of modern trace analysis, for example for diagnostic applications.

To achieve even lower detection limits, luminescence-based methods appear more suitable, because of the greater selectivity of signal generation. In this arrangement, luminescence excitation is limited to the penetration depth of the evanescent field into the medium of lower refractive index, i.e. to the immediate proximity of the waveguiding area, with a penetration depth of the order of some hundred nanometers into the medium. This principle is called evanescent luminescence excitation.

In combination with luminescence detection, the sensitivity has been increased considerably in recent years by means of highly refractive thin-film waveguides, based on a waveguiding film only a few hundred nanometers thick on a transparent support material. In WO 95/33197, for example, a method is described wherein the excitation light is coupled into the waveguiding film by a relief grating as a diffractive optical element. The isotropically emitted luminescence from substances capable of luminescence, which are located within the penetration depth of the evanescent field, is measured using suitable measurement arrangements, such as photodiodes, photomultipliers or CCD cameras. The portion of evanescently excited radiation that has backcoupled into the waveguide can also be outcoupled by a diffractive optical element, such as a grating, and be measured. This method is described, for example, in WO 95/33198.

In the last few years, new developments of planar thin-film waveguides as sensing platforms for “microarrays”, in some case combined with appropriately adapted fluidic structures, have become known, for example in the international patent applications WO 00/75644, WO 00/113096, WO 00/143875, which are fully incorporated in this application. In WO 01/79821 a thin-film waveguide structure is described, which enables a two-photon excitation on the surface of the waveguide. In WO 01/88511, a grating waveguide structure and a measurement method based thereon are described, which provide an imaging method for analyte determination based on a refractive measurement method. Both disclosures are also incorporated as parts of this patent application.—It is common to the above mentioned arrangements that biological or biochemical or synthetic recognition elements for the determination of a multitude of analytes in each case are immobilized in discrete measurement areas of known location, as parts of one or more arrays of measurement areas, on a supporting substrate.

Surprisingly, it has now been found that, with a suitable selection of the physical-chemical parameters of an evanescent field sensor platform (such as layer thicknesses, refractive indices of the involved layers), the achievable sensitivity for the detection of molecular interactions on the surface of the evanescent field sensor platform is sufficiently high, as a result of the high excitation light intensity at its surface and the simultaneous confinement of that strong excitation field to the penetration depth of the evanescent field into the adjacent media, for analyzing multiple samples or dilutions of these samples for the analytes contained therein after direct deposition of said samples or of their dilutions on said evanescent field sensor platform, without their prior isolation from the residual sample matrix. Thus, a method with an inverted assay architecture is enabled, wherein a sample to be deposited in a measurement area on the evanescent field sensor platform as a solid support has “unchanged relative molecular composition” (according to the definition given above), when compared with the “original sample”.

In the spirit of this invention, a “nature-identical” sample shall be attributed to a mixture of compounds to be analyzed, which has the same relative molecular composition (“unchanged relative molecular composition” according to the definition given above) of analytes to be determined therein as the original sample from which it is obtained. According to this definition, the original sample will also be a “nature- identical” sample. The original sample may, for example, be a biological cell comprising different molecules or compounds in a heterogeneous distribution. A sample obtained from a body of one or more cells selected beforehand from a larger number of cells, for example by centrifugation, filtration or laser capture micro dissection, shall also be referred to as an “original sample”.

In the following, the designation of a (single) cell for the sample preparation steps to be performed also refers in each case to a multiplicity of cells, unless explicitly stated otherwise.

In a first preparation step, which is typically necessary for further analysis steps, the cell may be lysed. The derived lysate with a homogeneous distribution of the compounds contained therein shall also be called a “nature-identical” sample, if the relative molecular composition of the analytes to be determined therein has remained unchanged. In particular, it is characteristic of a “nature-identical” sample that it contains the whole proteome of the “original sample”. The lysate may be dissolved in a suitable solvent, such as a buffer solution, and may contain known additives, for example stabilizers such as enzyme inhibitors, in order to prevent a digestion of the biopolymers contained therein. A “nature-identical” sample according to the definition may also contain known concentrations of compounds (as standards) similar to the analytes to be determined as additives, comparable with “spiking” of samples in chromatography. Such additives may, for example, be used for calibration purposes. Further on the “nature-identical” samples may contain additives of compounds similar to the sample matrix, such as bovine serum albumin (BSA), but different from the analytes to be determined, which may, for example be used for establishing a controlled surface density of immobilized analyte molecules in a measurement area. Analytes, i.e., especially biopolymers such as nucleic acids or proteins contained in the “nature-identical” samples may be present in native or in denatured form, for example after treatment of the “original sample” with urea or surfactant (e.g. SDS).

The analytes, i.e. especially biopolymers such as nucleic acids or proteins contained in the “nature-identical” samples are preferably present in denatured form, after treatment with urea, whereas the epitopes of the contained analytes are freely accessible for the binding to their corresponding detection reagents, such as antibodies. This is made possible by the destruction of the tertiary and quarternary structure due to the treatment with urea.

Surprisingly, the sensitivity of the method according to the invention is such that a “nature-identical” sample may even be highly diluted, and compounds contained in the mixture, in spite of their very low concentration in some cases and correspondingly small available amount in a single measurement area, can still be determined with high precision, which is not possible with the known conventional methods. This has the significant advantage that, in case of the method according to the invention, the deposited analytes which are contained in the sample and are to be determined are generally still present in the same relative molecular composition as in the original sample even after their immobilization.. The method according to the invention can thus provide analysis results which are representative of the overall molecular composition of the original sample, because the otherwise typical enrichment and separation steps can be avoided.

In the spirit of this invention, a molecular species or compound which can be distinguished from different compounds contained in a sample to be analyzed and can be bound by a specific detection reagent applied for this purpose shall be called an “analyte”. If, for example, only the phosphorylated, but not the nonphosphorylated form of a compound or species to be detected, these two forms of a compound or species correspond to two different analytes according to this definition. If any phosphorylated compounds or species are recognized and bound by another detection reagent, then, under these conditions, the corresponding phosphorylated compounds or species together are one analyte. According to this definition, specific binding partners as tracer compounds for an analyte may be selected, for example, in such a way that they exclusively recognize and bind to the phosphorylated or the glycosylated (or correspondingly to the nonphosphorylated and/or nonglycosylated) form of a compound to be detected. The activity of a biological signal pathway in a cell or organism may be correlated with the fraction of phosphorylated or glycolysated compounds (depending on the nature of the signal pathway) which control the corresponding signal pathway. The relative fraction of the phosphorylated and the glycolysated form, respectively, within the whole amount of the corresponding compound, i.e., the ratio of the amount of a compound present in its phosphorylated and its glycolysated form, respectively, and of the whole amount of this compound present in phosphorylated and nonphosphorylated form or in glycolysated and not glycolysated form, respectively, shall be called in the following the degree of phosporylation and the degree of glycolysation, respectively, of the corresponding compound in the sample. The degree of phosphorylation and the degree of glycolysation shall be summarized under the generic term of the “degree of activation” of a compound. However, the degree of activation of a compound may also mean other, chemically modified forms of a compound.

Specific binding partners as tracer compounds may also be selected in such a way that they only bind to a compound to be detected if this compound is present in a certain three-dimensional structure. For example, many antibodies only recognize and bind to specific partial regions (epitopes) of a compound to be determined when they are provided in a special three-dimensional structure. Depending on the conformational state of the compound to be determined, these partial regions (epitopes) may be accessible for the binding of the corresponding tracer compounds or may be hidden. The specific binding partners may also be selected in such a way that they bind to regions of the compound to be detected, the accessibility of these regions being independent of the three-dimensional structure of the corresponding compound. Through the use of appropriately selected tracer compounds it is thus possible to determine the relative amount of the total quantity of a compound which is to be detected in a sample and which shows a specific conformational state.

Such compounds which are known to be involved in specific binding reactions with molecules or compounds of biological origin or with their synthetically produced analogues shall be called “biologically relevant”. Examples of “biologically relevant” compounds are thus not only naturally occurring proteins, such as antibodies or receptors, or nucleic acids, but also their binding partners, such as antigens, which may be synthetic compounds even of very low molecular weight.

In the spirit of the present invention, spatially separated or discrete measurement areas shall be defined by the closed area that is occupied by binding partners immobilized thereon, for determination of one or more analytes in one or more samples in a bioaffinity assay. These areas may have any geometry, for example the form of circles, rectangles, triangles, ellipses etc.

Various such measurement areas may, for example, comprise a multitude of different samples deposited on the supporting substrate, or they may comprise different deposited dilutions of one or more samples (always deposited in different discrete measurement areas). The material for the deposition in the discrete measurement areas may also be provided for example by selective micro preparations, such as selective capture of individual cells from a cellular assembly by “laser capture micro dissection”.

More generally, the “nature-identical” sample with the analytes to be determined therein may be selected from the group comprising extracts of healthy or diseased cells (for example, of human, animal, bacterial or plant cell extracts), extracts of human or animal tissue, such as organ, skin, hair or bone tissue, or of plant tissue, and body fluids or their constituents, such as blood, serum or plasma, synovial fluid, lacrimal fluid, urine, saliva, tissue fluid, lymph. A “nature-identical” sample may in particular also be selected from the group comprising extracts of simulated (treated) or untreated cells and extracts of healthy and diseased tissue.

Accordingly, a “nature-identical” sample may also be taken from an organism or tissue or cellular assembly or cell by means of a method of the group of tissue slicing or biopsy, as well as by laser capture micro dissection.

In general, several different binding partners will be immobilized simultaneously in one measurement area in general. Typically, there will be multiple, i.e. several hundred or even several thousand, different analytes immobilized in one measurement area.

A first subject of the invention is a method for the analysis of multiple “nature-identical” samples for analytes which are contained therein and are of biological relevance as binding partners in specific binding reactions, wherein
said samples or dilutions of said samples of the same relative molecular composition as the original samples, with the analytes to be determined contained therein, as a first plurality of specific binding partners, are deposited in discrete measurement areas in one or more one- or two-dimensional array of measurement areas on an evanescent field sensor platform as a solid support without a change in the relative molecular composition, compared with the original relative molecular composition of the sample,
one or more tracer compounds as a second plurality of specific binding partners, for the specific determination of analytes from the first plurality of specific binding partners contained in the samples, are brought into contact with the samples deposited in said discrete measurement areas in a single step or multiple steps of a specific binding reaction,
changes in opto-electronic signals, resulting from the binding of tracer compounds to analytes contained in the samples in discrete measurement areas in the evanescent field of the evanescent field sensor platform are measured laterally resolved, and
the presence of the analytes to be specifically detected is determined qualitatively and / or quantitatively from the relative magnitude of the changes in said opto-electronic signals from the corresponding measurement areas.

The simplest method for immobilizing the specific binding partners for an analyte determination in a specific binding reaction is physical adsorption, for example, based on hydrophobic interactions between the specific binding partners to be immobilized and the evanescent field sensor platform as the solid support. The strength of these interactions, however, may be markedly changed by the composition of the medium and its physical / chemical properties, such as polarity and ionic strength. Especially in the case of the sequential supply of different reagents in a multi-step assay, the adhesion of the recognition elements is often insufficient after purely adsorptive immobilization on the surface. It is therefore preferred if the evanescent field sensor platform comprises an adhesion-promoting layer on which the samples are deposited, in order to improve the adhesion of the “nature-identical” samples or of their dilutions deposited in discrete measurement areas.

The adhesion-promoting layer has a thickness of preferably less than 200 nm, especially preferably less than 20 nm.

Various materials are suitable for generating the adhesion-promoting layer. For example, the adhesion-promoting layer may comprise compounds of the group of silanes, functionalized silanes, epoxides, functionalized, charged or polar polymers and “self-organized passive or functionalized mono- or multi-layers”, thiols, alkyl phosphates and alkyl phosphonates, multi-functional block copolymers, such as poly(L)lysin/polyethylene glycols.

Said adhesion-promoting layer may also comprise compounds of the group of organophosphoric acids of the general formula I (A)
Y—B—OPO_{3 l H}₂ (IA)
or of organophosphonic acids of the general formula I (B)
Y—B—PO₃H₂ (IB)
and of their salts, wherein B is an alkyl, alkenyl, alkinyl, aryl, aralkyl, hetaryl, or hetarylalkyl residue, Y is hydrogen or a functional group of the following series, e.g. hydroxy, carboxy, amino, mono- or dialkyl amino optionally substituted by lower alkyl, thiol, or negative acidic group of the following series, e.g. ester, phosphate, phosphonate, sulfate, sulfonates, maleimide, succinimydyl, epoxy or acrylate. These compounds have been described in more detail in the international patent application PCT/EP 01/10077, which is hereby incorporated in this disclosure in its whole entirety.

The method according to the invention is preferably designed in such a way that the relative molecular composition of a first plurality of specific binding partners as analytes immobilized in a measurement area is identical with the original relative molecular composition of the sample applied to said measurement area. This requirement may, for example, be met if the material amount of a sample deposited in a measurement area is equal to or less than the amount of material necessary for the formation of a monolayer on the evanescent field sensor platform as a solid support. At the same time, an optimum accessibility of the first plurality of immobilized specific binding partners as analytes for the tracer reagents to be brought into contact with them is given in case of a sub-monolayer coverage of the surface of the solid support. The accessibility may be even further improved if the adhesion-promoting layer deposited beforehand leads to an oriented immobilization, for example if antibodies contained in the deposited sample are immobilized bound to their F_{C}-part, resulting in accessibility of their specific binding epitopes.

Because of the high sensitivity of the method according to the invention, it is possible to analyze even very small volumes and quantities of sample used. The quantity of sample here shall be taken to mean the total quantity of material which is deposited in a discrete measurement area. A sample may, for example, comprise the material of less than 20000 cells and still be analyzed with high precision. A sample to be deposited may even comprise the material of less than 1000 cells. The required sample amount may even comprise the material of less than 100 cells, or even the material of only 1-10 cells, and still be analyzed reliably. The material corresponding to the content of a single cell shall be also called a cell-equivalent. An amount of cell-equivalents such small necessary for an analysis is required when the analytes to be detected are contents of relatively high abundance. It is also possible that a sample has a volume of less than 1 μl. A sample to be may even have a volume of less than 10 nl or even less than 1 nl.

The method according to the invention allows to determine the relative total amounts of one or more compounds contained as analytes in a “nature-identical” sample, as the sum of their occurrence in phosphorylated or not phosphorylated form and/or glycolisated and/or not glycolisated form. It is preferable if the relative amounts of one or more compounds contained as analytes in a “nature-identical” sample, in each case of their occurrence in phosphorylated and/or nonphosphorylated form and/or glycolysated and/or nonglycolysated form, are determined for one or more said forms.

The method according to the invention allows the degree of activation, as defined above, of one or more analytes contained in a “nature-identical” sample to be determined. In particular, the method according to the invention allows the degree of phosphorylation and/or the degree of glycolysation of one or more analytes contained in a “nature-identical” sample to be determined. As a result of the high sensitivity and high precision and reproducibility, in particular as a result of the numerous independent referencing and calibration methods that can be applied simultaneously or alternatively, it is also characteristic of the method according to the invention that differences of less than 20%, preferably less than 10%, between the relative amounts of one or more compounds contained in phosphorylated and/or nonphosphorylated and/or glycolysated and/or nonglycolysated form as analytes in a “nature-identical” sample and in one or more comparison samples can be determined for one or more of said forms.

As a result of the inherent, method-specific high sensitivity and the diversity of possibilities for referencing and/or calibration using one and the same analytical platform (evanescent field sensor platform), it is an important advantage of the method according to the invention that the variation of the measurement results obtained with this method is very low. The method according to the invention is thus also suitable for investigating the temporal evolution (i.e. the changes) of the relative amounts or concentrations of biologically relevant compounds influenced by a disease of a biological organism or of a cell culture and/or upon external manipulation of an organism or a cell culture.

It is therefore characteristic of another embodiment of the method according to the invention that said “nature-identical” sample and one or more comparison samples are taken from the same source of origin at different times, and that temporal changes of the relative amounts of one or more compounds in phosphorylated and/or nonphosphorylated form and/or glycolysated and/or nonglycolysated form contained as analytes in these samples are determined. “The same source of origin” shall here mean the same organism or an organism of similar type or the same cell culture of a cell culture of similar type (in each case after similar disease or manipulation of different duration). It is preferred if the method according to the invention allows temporal changes of less than 20%, preferably less than 10%, in the relative concentration and/or amount of said analytes to be determined.

If the original concentration of the analytes contained in a sample is sufficiently high, said samples can be dissolved and/or diluted in a liquid dilution medium, before their deposition on said evanescent field sensor platform as a solid support, and different dilutions of a sample can then be deposited in different discrete measurement areas on said evanescent field sensor platform. In this case, said samples can be diluted by at least a factor of 10 or even by a factor of 30 or 100.

Different samples may be taken from the same organism or the same cell culture. Then, for example, statistical information about the reproducibility of the relative molecular composition of the samples deposited in different measurement areas may be obtained through analysis of the materials contained on these measurement areas and derived from the same organism or from similar cell cultures (or from the same cell culture).

Different samples may in particular be taken from different positions of the same organism. Then, for example, information can be obtained about inhomogeneities of the relative molecular composition of the analytes to be determined in the organisms, from where said samples have been taken, from the analyses on the corresponding discrete measurement areas. Such a procedure is, for example, of great importance for the examination of cancerous organisms.

However, different samples can also be taken from different organisms or different cell cultures. For example, the samples may be taken from organisms that have been treated with a pharmaceutical drug and from those that have not been treated. The effect of the drug in question on the relative molecular composition of the samples can then be investigated in a manner similar to that of expression analysis in nucleic acid analytics.

A special embodiment of the method according to the invention comprises one or more samples being mixed with a solution of polymers or polymerizable monomers, optionally in the presence of initiators, or of chemical cross-linkers (e.g. glutaraldehyde), prior to their deposition on the evanescent field sensor platform as a solid support (in order to improve their adhesion on said solid support and to improve the homogeneity of the deposition). This embodiment of the method may, for example, help to avoid the formation of inhomogeneities of the distribution of the sample material within a measurement area during the evaporation process of the sample liquid, resulting in a better “spot morphology” and thus facilitating analysis of the results. It is preferred if said solution of polymers, polymerizable monomers or chemical cross-linkers is selected from the group comprising solutions of polysaccharides, such as agarose, acrylamides, glutaraldehyde etc.

It is also characteristic of this special variant of the method according to the invention that the mixture of the one or more samples with a solution of polymers or polymerizable monomers, optionally in the presence of initiators, or of chemical cross-linkers (e.g. glutaraldehyde), leads to immobilization of a three-dimensional network structure on the evanescent field sensor platform as a solid substrate, with sample components embedded therein, which are accessible for tracer reagents in the consecutive step of a bioaffinity reaction. Thus a higher degree of surface coverage of the evanescent field sensor platform than a monolayer can be achieved, which may lead to a further increase in the measurable signals in the analyte detection step. It is important here that the polymeric network structure which is generated does not extend beyond the penetration depth of the evanescent field into the medium, as an analyte detection is not possible beyond this distance from the surface of the evanescent field sensor platform and it would thus not be guaranteed that the result of the analysis would correspond to the original relative molecular composition of the sample.

The samples may be deposited with lateral selectivity in discrete measurement areas, either directly on the evanescent field sensor platform or on an adhesion-promoting layer deposited thereon, by means of a method selected from the group of methods comprising ink jet spotting, mechanical spotting by pen, pin or capillary, “micro contact printing”, fluidic contacting of the measurement areas with the samples through their supply in parallel or crossed micro channels, with the application of pressure differences or electrical or electromagnetic potentials, and photochemical or photolithographic immobilization methods.

It is of advantage if regions between the discrete measurement areas are “passivated” in order to minimize nonspecific binding of tracer compounds, i.e. that compounds which are “chemically neutral” (i.e. nonbinding) towards the analytes and the other contents of the deposited samples and the tracer compounds for said analytes are deposited between the laterally separated measurement areas.

Said compounds which are “chemically neutral” (i.e. nonbinding) towards the analytes and the other contents of the deposited samples and the tracer compounds for said analytes may be selected from the group comprising albumins, especially bovine serum albumin or human serum albumin, casein, nonspecific, polyclonal or monoclonal, heterologous or empirically nonspecific antibodies (for the analytes to be determined, especially for immunoassays), detergents—such as Tween 20—, fragmented natural or synthetic DNA not hybridizing with polynucleotides to be analyzed, such as extracts of herring or salmon sperm, or also uncharged but hydrophilic polymers, such as polyethyleneglycols or dextrans.

Without loss of generality, the analytes which are to be determined and are contained in the samples deposited in discrete measurement areas may be compounds of the group comprising, for example, proteins, such as monoclonal or polyclonal antibodies and antibody fragments, peptides, enzymes, glycopeptides, oligosaccharides, lectins, antigens for antibodies, proteins functionalized with additional binding sites (“tag proteins”, such as “histidine tag proteins”) and nucleic acids (e.g. DNA, RNA). The analytes which are to be determined and are contained in the samples deposited in discrete measurement areas may also be compounds of the group comprising cytosolic or membrane-bound cell proteins, especially proteins, such as kinases, which are involved in processes of signal transduction in cells. The analytes may also be biotechnologically modified polymers, e.g. biologically expressed bioloymers comprising luminescent or fluorescent groups, respectively, such as “blue fluorescent proteins” (BFP), “green fluorescent proteins” (GFP), or “red fluorescent proteins” (RFP).

Depending on the physical design of the evanescent field sensor platform, there are several possibilities for the metrological type of signal generation in analyte determination. A characteristic of one possible variant is that, as a consequence of the binding of tracer compounds to analytes contained in the “immobilization samples” in discrete measurement areas, the changes in opto-electronic signals which are to be determined in a laterally resolved manner are caused by local changes in the resonance conditions for the generation of surface plasmons in a thin metal layer as part of said evanescent field sensor platform.

As techniques of measurement, the resonance angle (upon variation of the incidence angle of the irradiated light at constant wavelength) and the resonance wavelength (upon variation of the irradiated excitation wavelength at constant incidence angle) can be measured for the determination of changes in the resonance conditions. Consequently, said change in the resonance conditions may be manifested by a change in the resonance angle for the irradiation of an excitation light for generation of a surface plasmon in a thin metal layer as part of said evanescent field sensor platform. Accordingly, said change in the resonance conditions may also be manifested by a change in the resonance wavelength of an irradiated excitation light for generation of a surface plasmon in a thin metal layer as part of said evanescent field sensor platform.

As a consequence of the binding of tracer compounds to analytes contained in the samples in discrete measurement areas, the changes in opto-electronic signals to be determined in laterally resolved manner may be caused by local changes in the effective refractive index in these regions on said evanescent field sensor platform.

Another important embodiment of method according to the invention comprises the changes in opto-electronic signals which are to be determined laterally resolved, as a consequence of the binding of tracer compounds to analytes contained in the samples in discrete measurement areas, being caused by local changes in one or more luminescences from molecules capable of luminescence, which are located within the evanescent field of said evanescent field sensor platform.

It is preferred if said changes in one or more luminescences originate from molecules or nanoparticles capable of luminescence, which are bound as luminescence labels to one or more tracer compounds for the analytes contained in discrete measurement areas.

It is especially advantageous if two or more luminescence labels with different emission wavelengths and/or different excitation spectra, preferably with different emission wavelengths and identical excitation wavelength, are applied for analyte detection. If several luminescence labels with different spectral properties, especially with different emission wavelengths, are bound to different detection reagents of the second plurality of specific binding partners which are brought into contact with the measurement areas, for example, different analytes can be determined in a single detection step, i.e. when the measurement areas are brought into contact with said detection reagents and the generated luminescences are detected simultaneously or consecutively.

Such a variant of the method according to the invention is, for example, especially suitable for simultaneously detecting, for example, the phosphorylated and the nonphosphorylated form of a compound, especially also within one (common) measurement area, by using two correspondingly different specific binding partners as tracer compounds, which are in this case directly labeled (e.g. with green and red emitting luminescence labels, respectively).

In a similar way, two or more analytes can be detected simultaneously if two or more luminescence labels with different emission decay times are applied for analyte detection.

For the method according to the invention, it is therefore preferred if two or more luminescence labels are applied for detecting different analytes in a sample. It is also preferred if two or more luminescence labels are applied for detecting different analytes in a measurement area.

It is also advantageous if the excitation light is irradiated in pulses with a duration between 1 fs and 10 minutes, and the emission light from the measurement areas is measured in a time-resolved manner.

The evanescent field sensor platform, as a solid substrate, preferably comprises an optical waveguide, comprising one or more layers. This may, for example, be a fiberoptic waveguide comprising several layers. Preferably however, it is a planar optical waveguide, which is provided as a continuous surface of the evanescent field sensor platform or may also be partitioned in discrete waveguiding regions, as is described, for example, in patent application WO 96/35940, which is incorporated in its full entirety in the present application.

An especially preferred embodiment of the method according to the invention comprises the evanescent field sensor platform as a solid substrate comprising a planar optical thin-film waveguide with an essentially optically transparent waveguiding layer (a) on a second, likewise essentially optically transparent layer (b) with lower refractive index than layer (a), and optionally with a likewise essentially optically transparent intermediate layer (b) between layers (a) and (b), with likewise lower refractive index than layer (a).

The excitation light from one or more light sources may be in-coupled into a waveguiding layer of the evanescent field sensor platform using one or more optical in-coupling elements from the group comprising prism couplers, evanescent couplers comprising joined optical waveguides with overlapping evanescent fields, front face (butt) couplers with focusing lenses, preferably cylindrical lenses, arranged in front of a front face (distal end) of the waveguiding layer, and grating couplers.

It is preferred if the in-coupling of excitation light into a waveguiding layer of the evanescent field sensor platform is performed using one or more grating structures (c) that are formed in said waveguiding layer.

It is also preferred if the out-coupling of light guided in a waveguiding layer of an evanescent field sensor platform is performed using one or more grating structures (c′) which are formed in said waveguiding layer and have the same or different grating period and grating depth as grating structures (c).

An especially preferred embodiment of the method according to the invention comprises excitation light from one or more light sources being in-coupled into a waveguiding layer of said evanescent field sensor platform using one or more grating structures (c), directed as a guided wave towards measurement areas located on the evanescent field sensor platform, wherein furtheron luminescence from molecules capable of luminescence, which is generated in the evanescent field of said guided wave, is measured in a locally resolved manner using one or more detectors, and wherein the relative concentration of one or more analytes is determined from the relative intensity of these luminescence signals.

A special variant consists in changes in the effective refractive index on the measurement areas being determined in addition to the determination of one or more luminescences.

For a further improvement in sensitivity it can be advantageous here if the determinations of one or more luminescences and/or determinations of light signals at the excitation wavelength are performed as polarization-selective measurements. It is preferred here if the one or more luminescences are measured at a polarization that is different from the polarization of the excitation light.

Another subject of the present invention is an analytical platform for the analysis of multiple “nature-identical” samples for analytes which are contained therein and are of biological relevance as binding partners in bioaffinity reactions, comprising
an evanescent field sensor platform as a solid substrate
at least one one- or two-dimensional array of discrete measurement areas with binding partners for the determination of said analytes in a bioaffinity reaction, immobilized in said measurement areas on the evanescent field sensor platform,
   wherein said discrete measurement areas are generated by deposition of said “nature-identical” samples or of dilutions derived therefrom, of the same relative molecular composition as the original samples, containing the analytes to be determined as a first plurality of specific binding partners, and wherein the one or more immobilized binding partners forming the first plurality of specific binding partners are the one or more analytes themselves contained in said “nature-identical” samples.

Proteins contained in the “nature-identical” samples may be present in native or in denatured composition, for example, after treatment of the “original sample” with urea or surfactants (e.g. SDS).

The “nature-identical” samples are preferably present in denatured form, after treatment with urea, whereas the epitopes of the analytes contained therein are freely accessible for binding to their corresponding detection reagents, such as antibodies. This is made possible by destruction of the tertiary and quarternary structure due to the treatment with urea.

It is preferred if the evanescent field sensor platform comprises an adhesion-promoting layer, on which the samples or fractions or dilutions thereof are deposited, in order to improve the adhesion of the “nature-identical” samples or of their dilutions deposited in discrete measurement areas.

The thickness of the adhesion-promoting layer here is preferably less than 200 nm, especially preferably less than 20 nm.

The adhesion-promoting layer may comprise compounds of the group of silanes, functionalized silanes, epoxides, functionalized, charged or polar polymers and “self-organized passive or functionalized mono- or multi-layers”, thiols, alkyl phosphates and alkyl phosphonates, multi-functional block copolymers, such as poly(L)lysin/polyethylene glycols.

It has been found to be especially advantageous if said adhesion-promoting layer comprises compounds of the group of organophosphoric acids of the general formula I(A)
Y—B—OPO₃H₂ (IA)
or of organophosphonic acids of the general formula I(B)
Y—B—PO₃H₂ (IB)
and of their salts, wherein B is an alkyl, alkenyl, alkinyl, aryl, aralkyl, hetaryl, or hetarylalkyl residue, Y means hydrogen or a functional group of the following series, e.g. hydroxy, carboxy, amino, mono- or dialkyl amino optionally substituted by low alkyl, thiol, or negative acidic group of the series ester, phosphate, phosphonate, sulfate, sulfonate, maleimide, succinimydyl, epoxy or acrylate.

It is preferred if the relative molecular composition of a first plurality of specific binding partners as analytes immobilized in a measurement area is identical to the original relative molecular composition of the sample applied to said measurement area.

A deposited “nature-identical” sample can be selected from the group comprising extracts of healthy or diseased cells (for example of human, animal, bacterial or plant cell extracts), extracts of human or animal tissue, such as organ, skin, hair or bone tissue, or of plant tissue, and comprising body fluids or their constituents, such as blood, serum or plasm, synovial liquids, lacrimal fluid, urine, saliva, tissue fluid, lymph.

In particular, a “nature-identical” sample may also be selected from the group comprising extracts of stimulated (treated) or untreated cells and extracts from healthy or diseased tissue.

Said samples may have been taken from an organism or tissue or cellular assembly or cell by means of a method of the group of tissue slicing, biopsy and laser capture micro dissection.

A deposited sample may comprise the material of less than 20000 cells or even of less than 1000 cells. The sample may have a volume of less than 1 μl or even of less than 10 nl.

The required sample amount may even comprise the material of less than 100 cells and still be analyzed reliably. This is the case when the analytes to be detected are ingredients occurring in a relatively high concentration.

One or more of said “nature-identical” samples may have been taken from biological organisms or tissue or cell assemblies or cells and been deposited directly (i.e. after lysis of the cells) on said solid support, without further dilution.

It is also possible that one or more of said “nature-identical” samples have been dissolved and/or diluted in a liquid dilution medium, before their deposition on said evanescent field sensor platform as a solid support, and wherein different dilutions of a sample have been deposited in different discrete measurement areas on said evanescent field sensor platform. Thereby, the samples can have been diluted by at least a factor of 10. The high sensitivity of the analytical platform according to the invention even allows the samples to be diluted by a factor of 30 or even 100 and a plurality of analytes within a single measurement area still to be quantitatively determined in spite of this marked dilution.

Different deposited samples may have been taken from the same organism or from the same cell cultures. In this case the samples may have been taken from different positions on the same organism.

Different deposited samples may also have been taken from different organisms or from different cell cultures.

A special embodiment of an analytical platform according to the invention comprises one or more samples being mixed with a solution of polymers or polymerizable monomers, optionally in the presence of initiators or of chemical cross-linkers (e.g. glutaraldehyde), prior to their deposition on the evanescent field sensor platform as a solid support (in order to improve their adhesion on said solid support and to improve the homogeneity of the deposition). This embodiment of the method may, for example, help to avoid the formation of inhomogeneities of the distribution of the sample material within a measurement area during the evaporation process of the sample liquid, resulting in a better “spot morphology” and thus facilitating the analysis of the results. It is preferred if said solution of polymers, polymerizable monomers or chemical cross-linkers is selected from the group comprising solutions of polysaccharides, such as agarose, acrylamides, glutaralehyde etc.

It is also characteristic of such a special embodiment of an analytical platform according to the invention that the mixture of the one or more samples with a solution of polymers or polymerizable monomers, optionally in the presence of initiators, or of chemical cross-linkers (e.g. glutaraldehyde), leads to an immobilization of a three-dimensional network structure on the evanescent field sensor platform as a solid substrate, with sample components embedded therein, which are accessible for tracer reagents in the consecutive step of a specific binding reaction. Thus a higher degree of surface coverage of the evanescent field sensor platform than a monolayer can be achieved, which may lead to a further increase in the measurable signals in the analyte detection step. It is important here that the polymeric network structure generated does not extend beyond the penetration depth of the evanescent field into the medium, because an analyte detection is not possible beyond this distance from the surface of the evanescent field sensor platform and thus it would not be guaranteed that the result of the analysis would correspond to the original relative molecular composition of the sample.

An advantageous embodiment of an analytical platform according to the invention is one wherein an array comprises more than 50, preferably more than 500, most preferably more than 5000 measurement areas.

Each measurement area here may comprise an immobilized “nature-identical” sample or comparison sample which is similar to or different from the samples immobilized in other measurement areas.

The measurement areas of an array may be arranged in a density of more than 10, preferably more than 100, most preferably more than 1000 measurement areas per square centimeter.

A further advantageous embodiment of an analytical platform according to the invention comprises multiple arrays of measurement areas being provided on an evanescent field sensor platform as a solid support. In particular at least 5, preferably at least 50 arrays of measurement areas are provided on an evanescent field sensor platform as a solid support. It is especially advantageous if different arrays of measurement areas of such an embodiment of an analytical platform according to the invention are provided in different sample compartments. For example, the international patent applications WO 00/75644, WO 00/113096 and WO 00/143875 it describe how an evanescent field sensor platform which is suitable for an analytical platform according to the invention is combined as a base plate with an adequate mounting body for the formation of an adequate array of sample compartments, each dedicated to housing an array of measurement arrays.

Such an embodiment of an analytical platform according to the invention allows an experimental arrangement that may be called “multi-dimensional”: For example, in the rows and columns of an array, different samples, for example from different organisms (e.g. corresponding to the columns), may be deposited at different dilutions (e.g. corresponding to rows). Different arrays of measurement areas, in different sample compartments, may then be brought into contact with different second pluralities of specific binding partners in different arrays for the determination of different analytes. Obviously, such a variant of an analytical platform according to the invention allows an almost unlimited number of different experiments to be performed.

It is also advantageous if regions between the discrete measurement areas are “passivated” in order to minimize nonspecific binding of tracer compounds, i.e. that compounds which are “chemically neutral” (i.e., nonbinding) towards the analytes and the other contents of the deposited samples and the tracer compounds for said analytes are deposited between the laterally separated measurement areas.

Said compounds which are “chemically neutral” (i.e. nonbinding) towards the analytes and other contents of the deposited samples and towards the tracer compounds for said analytes may be selected from the groups comprising albumins, especially bovine serum albumin or human serum albumin, casein, nonspecific, polyclonal or monoclonal, heterologous or empirically nonspecific antibodies (for the analytes to be determined, especially for immunoassays), detergents—such as Tween 20—, fragmented natural or synthetic DNA not hybridizing with polynucleotides to be analyzed, such as extracts of herring or salmon sperm, or uncharged but hydrophilic polymers, such as polyethylene glycols or dextrans.

Without loss of generality, the analytes which are to be determined and are contained in the samples deposited in discrete measurement areas may be compounds of the group comprising proteins, such as monoclonal or polyclonal antibodies and antibody fragments, peptides, enzymes, glycopeptides, oligosaccharides, lectins, antigens for antibodies, proteins functionalized with additional binding sites (“tag proteins”, such as “histidine tag proteins”) and nucleic acids (e.g. DNA, RNA).

In particular, the analytes which are to be determined and are contained in the samples deposited in discrete measurement areas may also be compounds of the group comprising cytosolic or membrane-bound cell proteins, especially proteins, such as kinases, which are involved in processes of signal transduction in cells. The analytes may also be biotechnologically modified polymers, e.g. biologically expressed bioloymers comprising luminescent or fluorescent groups, respectively, such as “blue fluorescent proteins” (BFP), “green fluorescent proteins” (GFP), or “red fluorescent proteins” (RFP).

A special variant of an analytical platform according to the invention comprises the evanescent field sensor platform, as part of the analytical platform, comprising a thin metal layer, optionally on an intermediate layer with refractive index preferably <1.5, such as silicon dioxide or magnesium fluoride, located beneath, and wherein the thickness of the metal layer and of the optional intermediate layer are selected in such a way that a surface plasmon can be excited at the wavelength of an irradiated excitation light and/or of a generated luminescence.

It is preferred here if the metal is selected from the group comprising gold and silver. It is also preferred if the metal layer has a thickness between 10 nm and 1000 nm, preferably between 30 nm and 200 nm.

The evanescent field sensor platform, as a solid substrate, preferably comprises an optical waveguide, comprising one or more layers. This may, for example, be a fiberoptic waveguide comprising several layers. Preferably, however, it is a planar optical waveguide which is provided as a continuous surface of the evanescent field sensor platform or may also be partitioned in discrete waveguiding regions, as is described, for example in patent application WO 96/35940.

Especially preferred is such an embodiment of the analytical platform according to the invention, wherein the evanescent field sensor platform as a solid substrate comprises a planar optical thin-film waveguide with an essentially optically transparent waveguiding layer (a) on a second, likewise essentially optically transparent layer (b) with lower refractive index than layer (a) and optionally with a likewise essentially optically transparent intermediate layer (b) between layers (a) and (b), with likewise lower refractive index than layer (a).

An analytical platform according to the invention is preferably designed in such a way that a waveguiding layer of the evanescent field sensor platform is in optical contact with one or more optical coupling elements enabling the in-coupling of excitation light from one or more light sources into said waveguiding layer, said optical coupling elements being selected from the group comprising prism couplers, evanescent couplers comprising joined optical waveguides with overlapping evanescent fields, front face (butt) couplers with focusing lenses, preferably cylindrical lenses, arranged in front of a front face (distal end) of the waveguiding layer, and grating couplers.

It is especially preferred if one or more grating structures (c) with similar or different grating period and grating depth as grating structures (c) are provided in a waveguiding layer of the evanescent field sensor platform, allowing the out-coupling of light guided in said waveguiding layer.

It is also advantageous if one or more grating structures (c), with similar or different grating period and grating depth as grating structures (c) are provided in a waveguiding layer of the evanescent field sensor platform allowing the out-coupling of light guided in said waveguiding layer.

Further embodiments of evanescent field sensor platforms which are suitable as an analytical platform according to the invention are described, for example, in patent applications WO 95/33197, WO 95/33198 and WO 96/35940, which are also incorporated in their full entirety in the present invention.

A further subject of the invention is the use of a method according to the invention and/or of an analytical platform according to the invention for quantitative and/or qualitative analyses for the determination of chemical, biochemical or biological analytes in screening methods in pharmaceutical research, combinatorial chemistry, clinical and pre-clinical development, for real-time binding studies and the determination of kinetic parameters in affinity screening and in research, for qualitative and quantitative analyte determinations, especially for DNA and RNA analytics and for the determination of genomic or proteomic differences in the genome, such as single nucleotide polymorphisms, for the measurement of protein- DNA interactions, for the determination of control mechanisms for mRNA expression and for the protein (bio)synthesis, for the generation of toxicity studies and the determination of expression profiles, especially for the determination of biological and chemical marker compounds, such as mRNA, proteins, peptides or small-molecular organic (messenger) compounds, and for the determination of antibodies, antigens, pathogens or bacteria in pharmaceutical research and development, human and veterinary diagnostics, agrochemical product research and development, for symptomatic and pre-symptomatic plant diagnostics, for patient stratification in pharmaceutical product development and for the therapeutic drug selection, for the determination of pathogens, nocuous agents and germs, especially of salmonella, prions and bacteria, especially in food and environmental analytics.

In the following, the invention is further explained by examples of applications. The embodiments described herein do not imply any loss of generality.

### EXAMPLES

### 1. Analytical Platform

### 1.1. Evanescent Field Sensor Platform

As an analytical platform, an evanescent field sensor platform serves as a solid support with the dimensions of 14 mm width×57 mm length×0.7 mm thickness. The evanescent field sensor platform is provided as a thin-film waveguide, comprising a glass substrate (AF 45) and a 150 nm thin, highly refractive layer of tantalum pentoxide deposited thereon. Two surface relief gratings, in parallel to the length of the evanescent field sensor platform, are modulated in the glass substrate at a distance of 9 mm between each other (grating period: 318 nm, grating depth: 12 nm ±2 nm). These structures, which shall serve as diffractive gratings for the in-coupling of light into the highly refractive layer, are carried over into the surface of the tantalum pentoxide layer in the subsequent deposition of the highly refractive layer.

After careful cleaning of the evanescent field sensor platform, a monolayer of mono dodecyl phosphate (DDP), as an adhesion-promoting layer, is generated on the surface of the metal oxide layer by spontaneous self-assembly, upon precipitation from an aqueous solution (0.5 mM DDP). This surface modification of the initially hydrophilic metal oxide surface leads to a hydrophobic surface (with a contact angle of about 100° against water), on which multiple “nature-identical” samples shall be deposited, the “nature-identical” samples containing analytes, as specific binding partners for the analyte detection in a specific binding reaction, shall be deposited.

Six identical , each with 90 measurement areas (spots) arranged in 10 rows and 9 columns, are deposited on the evanescent field sensor platform provided with a hydrophobic adhesion-promoting layer, using an inkjet spotter (model BCA1, Perkin Elmer, Boston, Mass., USA). Each spot is generated by deposition of a single droplet of 280 pl volume on the chip surface.

### 1.2. Reagents and Generation of Arrays of Measurement Areas

Human T-cell cultures (Jurkat, DMZ # ACC282) are utilized for the detection of biologically relevant protein analytes in “nature-identical” samples. These cells are cultivated at 37° C. in a solution containing RPMI 1640, 10 % FCS (fetal calf serum), 2 mM glutamine, 50 U/ml penicillin, 50 μg/ml streptamycine (cell density at about 0.5−×10⁶−1.0×10⁶ cells/ml). Then the cells are incubated with antibodies, namely “mouse-anti-human-CD3” (mouse-α-human-CD3) and “mouse-anti-human-CD28” (mouse-(α-human-CD28) (each in a solution of 1 μg/ml; incubation for 10 min), against the surface receptors CD3 and CD28, respectively. A cell culture, which is similar to the one described above but not treated with antibodies, is used as a comparison sample and shall serve as a negative control in the analytical detection method. A further cell culture similar to the one first described, except for the treatment with antibodies, is treated for 180 min with staurosporine (concentration: 10 μM), which is a strong protease inhibitor.

Then the cell cultures treated as described above and the untreated cell cultures, respectively, are cooled to 4° C. and formed to pellets by centrifugation at a centrifugal force of 350×g (number of cells at about 10⁷).The cells here are simply separated from the medium, without damage to the cells. The supernatant is then decanted, and lysis buffer (7 M urea, 2 M thiourea, 4% CHAPS, 1% DTT, 4 mM spermidine and Complete (protease inhibitor, Roche AG, 1 tablet/50 ml) is added, the total protein concentration being adjusted to about 10 mg/ml. Thereby, all protein-containing cell components are denatured spontaneously and completely and solubilized.

The treatment with the aforesaid antibodies serves as a model system for co-stimulant activation of human T-cells (M. Diehn et al., “Genomic expression programs and the integration of the CD28 costimulatory signal in T cell activation”, Proceedings of the National Academy of Sciences 99 (2002) 11796-11801). The binding of said antibodies to cell membrane-bound receptors leads to a phosphorylation cascade with different associated signal pathways within the affected cells. The activity of a certain signal pathway can be detected here by determining the degree of phosphorylation of a corresponding key protein (as a so-called “marker protein”) or of its substrate, which shall be performed using an analytical platform according to the invention.

As a reference method for the method according to the invention, a Western blot analysis is performed.

The samples obtained by the preparation steps described above are again diluted by a factor of 10 to a total protein concentration of about 1 mg/ml, and are then deposited as “nature-identical” samples in discrete measurement areas for generating an array of measurement areas on the evanescent field sensor platform provided with the adhesion-promoting layer.

In addition to the measurement areas comprising deposited “nature-identical” samples, each microarray comprises additional measurement containing immobilized bovine serum albumin fluorescently labeled with Cy5 (Cy5-BSA), which are used for referencing local differences and/or temporal variations of the excitation light intensity (“reference spots”). Cy5-BSA (labeling rate: 3 Cy5 molecules per BSA molecule) is deposited at a concentration of 1.0 nM in phosphate-buffered sodium chloride solution (phosphate-buffered saline PBS, pH 7.4).

After deposition of the “nature-identical” samples and Cy5-BSA, the analytical platform is stored at ambient temperature and 100% relative humidity for two hours and then dried in ambient air. Then the free hydrophobic regions on the evanescent sensor platform not coated with protein are saturated with bovine serum albumin (BSA) by incubation of the surface with a solution of BSA (30 mg/ml) in 50 mM imidazole/100 mM NaCl (pH 7.4). The evanescent field sensor platform carrying the generated measurement areas is then washed with water, dried in a stream of nitrogen and stored at 4° C. until execution of the detection method according to the invention.

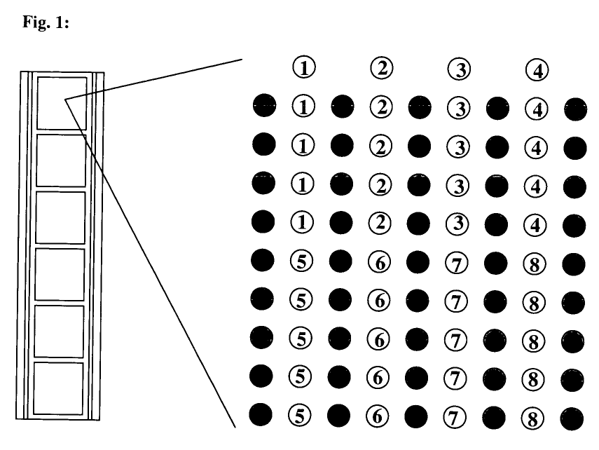

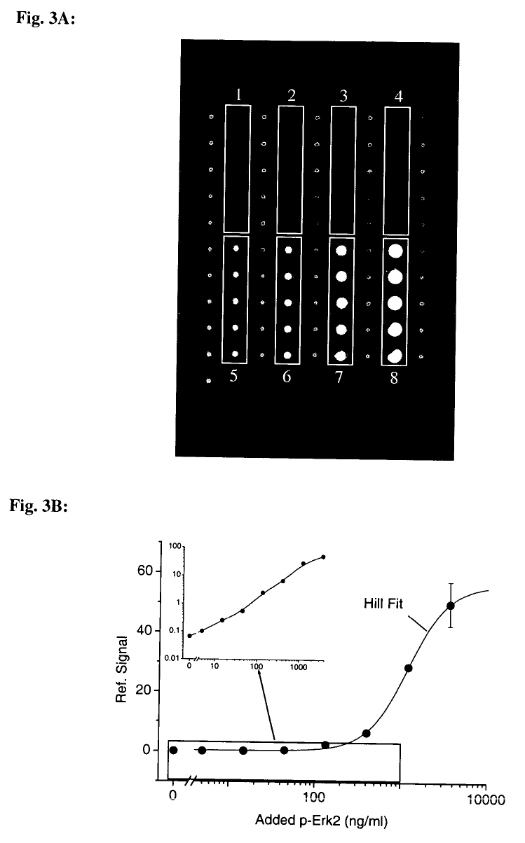

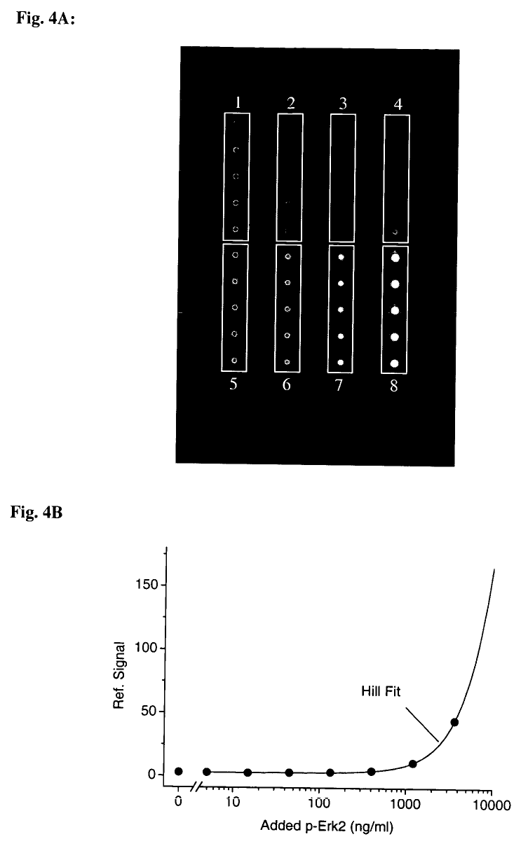

The geometry of a typical arrangement of measurement areas in a two-dimensional array and a linear arrangement of sic (identical) arrays on an evanescent field sensor platform is shown in FIG. 1 (for the examples which will be described in more detail with respect to FIG. 3A/B and FIG. 4A/B, respectively). The diameter of the spots, arranged at distance (center-to-center) of 600 μm, is about 90 μm. In the case of these examples, an array of measurement areas in each case comprises an arrangement of 8 different deposited samples with 5 replicates, the 5 similar measurement areas in each case being provided in a common column oriented perpendicular to the direction of propagation of the light guided in the waveguiding layer of the analytical platform during the detection step. The reproducibility of the measurement signals within the array of measurement areas shall be determined by means of the 5 similar measurement areas in each case. Columns of measurement areas containing deposited Cy5-BSA are arranged in each case between and beside the columns of measurement areas containing deposited samples to be analyzed (for purposes of referencing). In this example, the analytical platform according to the invention comprises 6 similar arrays of measurement areas of this kind, as shown in FIG. 1.

### 2. Analytical Detection Method

### 2.1. Assay Architecture

The detection of certain proteins in general form (i.e. for example with or without phsophorylation) and/or of certain proteins especially in activated (e.g. phosphorylated) form in the immobilized cell lysates as “nature-identical” samples deposited in discrete measurement areas is performed by sequential application of the corresponding detection reagents as assay steps before measurement of the resulting fluorescence signals: As a preparation for a first assay step, polyclonal analyte-specific rabbit antibodies (antibody A1 (#2261): phospho-(Ser) PKC substrate; antibody A2 (#9611): phospho-(Ser/Thr) Akt substrate; antibody A3 (#9101): Phospho-p44/42 MAP kinase (Thr202/Tyr204); antibody A4 (#9102): p44/42 MAP kinase (Thr202/Tyr204); all antibodies obtained from Cell Signaling Technology, INC., Beverly, Mass., USA) are typically diluted in a ratio of 1:500 in assay buffer (50 mM imidazole, 100 mM NaCl, 0.1% BSA, 0.05% Tween 20 pH 7.4). In each case, 30 μl of these four different antibody solutions are each applied to one of the six identical arrays of measurement areas, followed by an incubation at ambient temperature overnight (first assay step). Excess antibodies which are not specifically bound are removed by washing each array with assay buffer (5×100 μl).

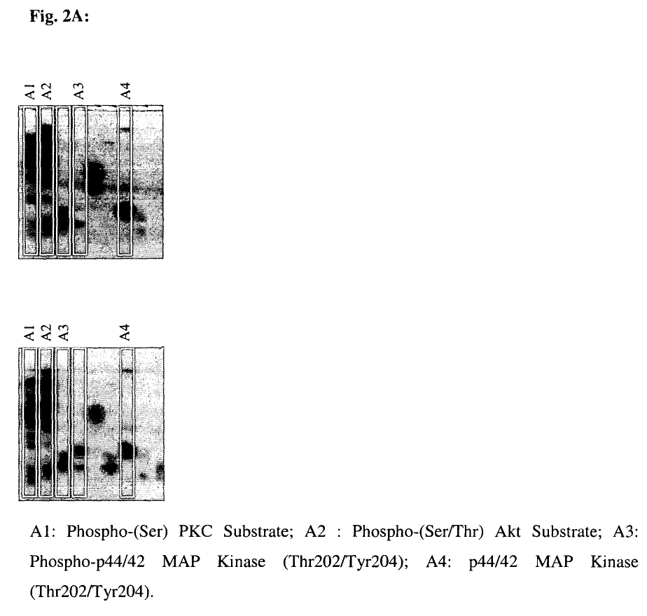

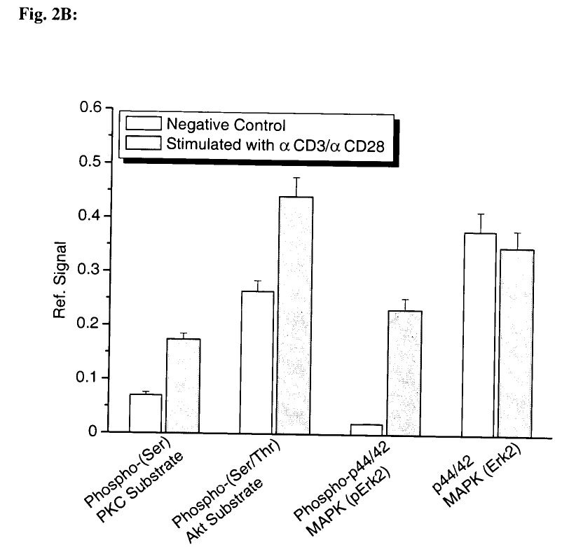

The 4 different antibodies used in this assay are basically different in nature: Antibodies A1 and A2 recognize and bind to different proteins phosphorylated at serine or serine/threonine, respectively, these protein kinases serving as substrates. This is discernible from the numerous bands in the Western blot (FIG. 2A and section 2.4, “Results”. Antibodies A3 and A4 recognize and bind to the same kind of compound, namely p44/42 MAP-kinase (also called Erk2), however, only antibody A3 recognizes its phosphorylated “activated” form (pErk2), whereas antibody A4 recognizes and binds to both forms (the not phosphorylated form Erk2 and the phosphorylated form pErk2).

A second assay step is performed for the detection of bound analyte-specific antibodies contained in discrete measurement areas comprising the immobilized “nature-identical” samples using a Cy5-labeled anti-rabbit antibody (Amersham Biosciences, Duibendorf, Switzerland), which binds to all aforesaid antibodies A1-A4. This Cy5-labeled antibody is applied to the arrays at a concentration of typically 10 nM in assay buffer (30 μl in each case), followed by an incubation for 2 hours in the dark at ambient temperature. Then the arrays are washed with assay buffer (five times each with 100 μl) in order to remove Cy5-anti-rabbit antibodies that are not specifically bound). The analytical platforms prepared in this way are then stored until execution of the detection step by means of excitation and detection of the resulting fluorescence signals using the ZeptoREADER™ (see below).

### 2.2. Detection of the Fluorescence Signals From the Arrays of Measurement Areas

The fluorescence signals from the various arrays of measurement areas undergo automatic sequential measurement using a ZeptoREADER™ (Zeptosens AG, Benkenstrasse 254, CH-4108 Witterswil). For each array of measurement areas, the analytical platform according to the invention is adjusted for matching the resonance condition for in-coupling of light into the waveguiding tantalum pentoxide layer and for maximizing the excitation light available in the measurement areas. Then, for each array, images of the fluorescence signals from the corresponding array are generated, wherein the user can select different exposure times and the number of images to be generated. In the case of measurements for the present example, the excitation wavelength is 633 nm, and the detection of the fluorescence light at the fluorescence wavelength of Cy5 is performed using a cooled camera, an interference filter (transmission 670 nm ±20 nm) for suppression of scattered light being positioned in front of the lens of the camera. The fluorescence images generated are automatically stored on the disk of the control computer. Further details of the optical system (ZeptoREADER™) are described in the international patent application PCT/EP 01/10012, which is incorporated in its entirety in the present application.

### 2.3. Evaluation and Referencing

The average intensity of the signals from the measurement areas (spots) is determined using an image analysis software (ZeptoVIEW, Zeptosens AG, CH-4108 Witterswil) enabling a semi-automatical analysis of the fluorescence images from a multitude of arrays of measurement areas.

The raw data of the individual pixels of the camera correspond to a two-dimensional matrix of digitized measurement data, corresponding to the imaged area on the sensor platform. For data analysis, first a two-dimensional coordinate grid is manually superimposed on the image points (pixels) in such a way that the image fraction of each spot is contained in an individual two-dimensional grid element. Within this grid element, an adjustable, circular “area of interest” (AOI) with a user-definable radius (typically 90 μm) is assigned to each spot. The location of the different AOIs is determined individually as a function of the signal intensity of the pixels by the image analysis software. The radius of the AOIs initially defined by the user is preserved. The arithmetic mean of the pixel values (signal intensities) within a chosen analysis area is determined as the mean gross signal intensity for each spot.

The background signals are determined from the signal intensities measured between the spots. For this purpose, four additional circular areas (typically with the same radius as the analysis areas of the spots) are defined as analysis areas for background signal determination for each spot, which are preferably located in the center between adjacent spots. The mean background signal intensity is, for example, determined as the arithmetic mean of the pixel values (signal intensities) within a defined AOI for each of the four circular areas. The mean net signal intensity from the measurement areas (spots) is then calculated as the difference between the mean local gross and background signal intensities of the corresponding spots.

Referencing of the net signal intensities of all the spots is performed by means of reference spots (Cy5-BSA) of each array of measurement areas. For this purpose, the net signal intensity of each spot is divided by the mean value of the net signal intensities of the adjacent reference spots within the same row of measurement areas (arranged in parallel with the direction of propagation of the light guided in the evanescent field sensor platform). This referencing method compensates for local differences in the available excitation light intensity along the direction perpendicular to the direction of light propagation, both within each microarray and between different microarrays.

### 2.4. Results

The results of the Western blot analysis as a comparison method for the method according to the invention are shown in FIG. 2A. The lower part of the figure shows the results obtained using cell cultures which had been treated with antibodies against the surface receptors CD3 (“αCD3”) and CD28 (“αCD28”), and the upper part of the figure shows the results obtained using the untreated cultures as comparison samples, in each case after their incubation with the aforesaid antibodies A1-A4.

It is confirmed by Western blot analysis that antibodies A1 and A2 recognize and bind to various proteins phosphorylated at serine or serine/threonine, respectively, these protein kinases serving as substrates. This is apparent from the multiple bands in the Western blot. Antibodies A3 and A4 recognize and bind to the same kind of compound, namely p44/42 MAP-kinase (also called Erk2); however, antibody A3 only recognizes its phosphorylated “activated” form (pErk2), whereas antibody A4 recognizes and binds to both forms (the nonphosphorylated form Erk2 and the phosphorylated form pErk2). It is therefore expected that a more intense band should develop after addition of A3 to the treated sample than after addition of A3 to the untreated sample (if any detectable amount of pErk2 is contained in this untreated sample at all). In fact, such a band is not visible in the Western blot of the untreated sample. For the treated sample, a single band is expected for each of the two cases. These expectations are completely confirmed by the results shown in FIG. 2A.

The results obtained with the method according to the invention using the analytical platform according to the invention are shown in FIG. 2B. The bar plot shows, for purposes of comparison, the results obtained with the cell culture treated with the antibodies against the surface receptors CD 23 (“αCD3”) and CD28 (“αCD28”) (solid¹ bars) and with the untreated culture (“negative control”), each of the “nature-identical” samples generated therefrom having been deposited in 6 similar arrays of measurement areas on an evanescent field sensor platform as described above and then brought into contact with the solutions of the different antibodies A1- A4. Different solutions, each containing one of the 4 aforementioned antibodies A1- A4, were each applied to 4 similar arrays arranged in different sample compartments on a common evanescent field sensor platform, and then Cy5-labeled anti-rabbit antibody was added, as described in section 2.1. For each case, the average values of the signal intensities, referenced according to the method described above and derived in each case from five similar measurement areas within an array, are shown in FIG. 2B together with their standard deviations.
In the original German text is written “leere Balken” (empty bars), which is an obvious error regarding FIG. 2A.

The signal intensities obtained correlate with the concentration of a certain analyte under consideration (high signal intensity corresponding to high concentration). It can be clearly seen that the relative intracellular concentration of phospo-(Ser) PKC substrates and phospho-(Ser/Thre) Akt substrates is increased by a factor of 2.5 and 1.8, respectively, in comparison with the negative control, resulting from the treatment (“stimulation”) of Jurkat cell cultures with antibodies against the surface receptors CD3 (“αCD3”) and CD28 (“αCD28”). The concentration of pErk2 was increased still more, namely by a factor of 10, whereas the sum of the contents of Erk2 and pErk2 (detected using antibody A4) remained constant, within the accuracy of measurement. This observation indicates that the total contents of Erk2 was not elevated by an increase in expression within the stimulation period of 10 minutes, but only the content of pErk2 was increased by phosphorylation. These results are in good agreement with the results from the Western blot analysis (FIG. 2A) performed as a comparison method, the increase of the fraction of phospho-(Ser/Thr) Akt substrates, however, being hardly discernible, if at all, in the case of Western blot analysis. In contrast to the results obtained using the method according to the invention, the Western blot analysis does not generally provide quantitative results with regard to the relative concentrations or their changes.

In order to evaluate the sensitivity of the method according to the invention for the detection of an individual “marker protein” of interest in a “nature-identical” sample deposited in a measurement area, i.e. within the proteome immobilized in an individual measurement area, an untreated cell lysate (negative control), which demonstrably contains only a very small amount of pErk2 (third pair of bars in FIG. 2B) according to the results of the assay performed before (results shown in FIG. 2B), was partitioned into individual aliquot solutions, to which this “marker protein” was added at different concentrations (0-3645 ng/ml). These solutions were then immobilized on the planar waveguide chip as described before, and an assay as described in section 2.1 was performed (using the antibody A3).

A typical distribution of the signals from an array of measurement areas is shown in FIG. 3A, the marked rectangles always indicating 5 replica spots with the untreated cell lysate to which a certain pErk2 concentration was added (1.8: increase in concentration, with the geometrical arrangement as shown in FIG. 1).

The result of this measurement for the detection of pErk2 as a function of its added concentration can be described by a typical binding curve, by fitting a Hill function to the concentration-dependent signal values (FIG. 3B). Each data point in FIG. 3B represents the mean value of the referenced net signal intensities from 5 replicate analyte spots, together with the standard deviation represented by error bars. The enlarged insert in FIG. 3B shows the concentration dependence of the signals at low concentrations, the increase of the values not being resolvable in the graphic representation for the whole concentration range. Based on the sum of the signal of the “0-value” (“blank”, i.e. sample without added pErk2) and its two-fold standard deviation, a value of 2.0 ng/ml, corresponding to a fraction according to weight of 2×10⁻⁶ g pErk2 in 1 g total protein, was determined as the sensitivity (limit of detection) of the assay.

FIGS. 4A and 4B show the results of a third assay essentially analogous to the second one. In contrast to the second assay described above, with the results shown in FIGS. 3A and 3B, this third assay is performed as described in section 2.1, using antibody A4 (i.e., upon application of this to similar arrays as used for the second assay). Thus, the total amount of the phosphorylated and the nonphosphorylated form of the compound (pErk2 and Erk2), corresponding to the differing amount of pErk2 added, is determined in this assay. A typical distribution of the signals from an array of measurement areas is shown in FIG. 4A, the marked rectangles in turn each representing 5 replica spots with the untreated cell lysate to which a certain pErk2 concentration was added (1.8: increase in concentration, with the geometrical arrangement as shown in FIG. 1). In this case, an assay sensitivity (limit of detection) of 120 ng/ml, corresponding to a fraction according to weight of 1.2×10⁻⁴ g pErk2 in 1 g total protein, is determined.

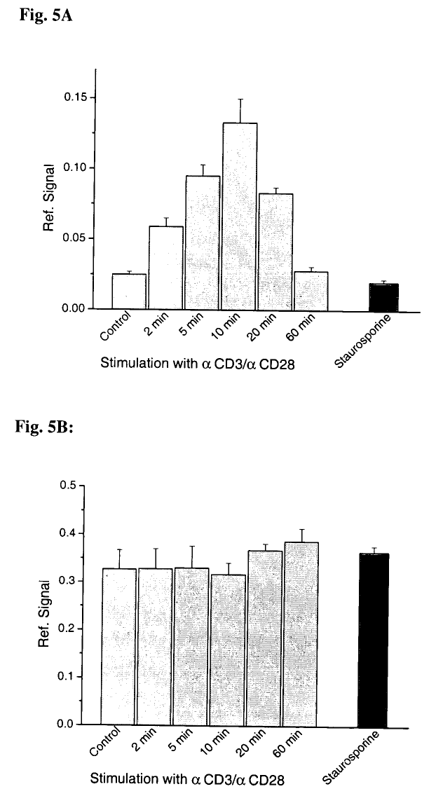

A further, fourth experiment is carried out to determine whether different changes can also be determined in the concentration of pErk2 resulting from co-stimulation of Jurkat cells by αCD3/αCD28 during stimulation periods of differing duration, and whether the differences in these changes can be resolved by the method according to the invention. For this purpose, Jurkat cell cultures are incubated in each case with 1 μg/ml αCD3/αCD28 for a different lengths of time (of the order of minutes) before lysis. Additionally, one Jurkat cell culture is treated with staurosporin (protein kinas inhibitor). The last-mentioned cell culture serves as a negative control, because no pErk2 should be present in this sample due to inhibition of all protein kinases. The signal measured for this sample should therefore correspond to the signal from a sample free from pErk2. The cell lysates treated as described above are then spotted onto the evanescent field sensor platform, and an assay as described in section 2.1 is performed, using antibody A3 to determine changes in the concentration of pErk2. The results of this measurement are shown in FIG. 5A. Each of the bars shown in this graph represents the referenced average value of the net signal intensities from 5 replicate analyte spots, together with the corresponding standard deviation. It can be clearly seen that changes in pErk2 concentration, detected using antibody A>3, can be readily resolved. The temporal dependence, i.e. the dependence on the length of the stimulation period, is characterized by a rapid increase in pErk2 concentration, followed by decrease to the level of the initial concentration after a stimulation period of 60 minutes, the concentration maximum being reached after about 10 minutes. The signal from the nonstimulated control sample is only slightly higher than the signal from the sample treated with staurosporin, the difference representing the natural content of pErk2 without stimulation.

As a control measurement, an assay and detection method are performed similar to the one just described, but using antibody A4 instead of antibody A3 to determine the total amount of the corresponding phosphorylated and nonphosphorylated protein form, i.e. the relative total content of Erk2/pErk2. This experiment does not show any significant signal differences, i.e. no changes in concentration, for the different stimulation periods of up to 60 minutes, and also no difference in comparison with the untrated control sample and the sample treated with staurosporin, within the experimental accuracy (FIG. 5B).

1. A method for the analysis of multiple “mature-identical” samples for analytes which are contained therein and are of biological relevance as binding partners in specific binding reactions, wherein
said samples or dilutions of said samples of the same relative molecular composition as the original samples, with the analytes which are to be determined and are contained therein, as a first plurality of specific binding partners, are deposited in discrete measurement areas in at least one one- or two-dimensional array of measurement areas on an evanescent field sensor platform as a solid support without a change in the relative molecular composition, compared with the original relative molecular composition of the sample,
one or more tracer compounds as a second plurality of specific binding partners, for the specific determination of analytes out of the first plurality of specific binding partners contained in the samples, are brought into contact with the samples deposited in said discrete measurement areas in a single step or multiple steps of a specific binding reaction,
changes in opto-electronic signals, resulting from the binding of tracer compounds to analytes contained in the samples in discrete measurement areas in the evanescent field of the evanescent field sensor platform are measured in a laterally resolved manner, and
the presence of the analytes to be specifically detected is determined qualitatively and/or quantitatively from the relative amount of the changes in said opto-electronic signals from the corresponding measurement areas.

2. A method according to claim 1, wherein the relative molecular composition of a first plurality of specific binding partners as analytes immobilized in a measurement area is identical with the original relative molecular composition of the sample applied to said measurement area.

3. A method according to claim 1, wherein the evanescent field sensor platform comprises an adhesion-promoting layer, on which the samples or their dilutions are deposited, in order to improve the adhesion of the “nature-identical” samples or of their dilutions deposited in discrete measurement areas.

4. A method according to claim 3, wherein the adhesion-promoting layer has a thickness of less than 200 nm, preferably of less than 20 nm.

5. A method according to claim 3, wherein said adhesion-promoting layer comprises compounds of the group of silanes, functionalized silanes, epoxides, functionalized, charged or polar polymers and “self-organized passive or functionalized mono- or multi-layers”, thiols, alkyl phosphates and alkyl phosphonates, multi-functional block copolymers, such as poly(L)lysin/polyethylene glycols.

6. (Deleted)

7. A method according to claim 1, wherein said “nature-identical” samples are selected from the group consisting of extracts of simulated (treated) or untreated cells and extracts of healthy or diseased tissue, extracts of healthy or diseased cells (for example of human, animal, bacterial or plant cell extracts), extracts of human or animal tissue, such as organ, skin, hair or bone tissue, or of plant tissue, and comprising body fluids or their constituents, such as blood, serum or plasm, synovial liquids, lacrimal fluid, urine, saliva, tissue fluid, lymph.

8. (Deleted)

9. A method according to claim 1, wherein said “nature-identical” samples have been taken from an organism or tissue or cellular assembly or cell by means of a method of the group comprising tissue slicing, biopsy and laser capture micro dissection.

10. (Deleted)

11. A method according to claim 1, wherein a sample comprises the material of less than 1000 cells.

12. A method according to claim 1, wherein analytes, i.e. especially biopolymers such as nucleic acids or proteins contained in a “nature-identical” sample are present in a native conformation.

13. A method according to claim 1, wherein analytes, i.e. especially biopolymers such as nucleic acids or proteins contained in a “nature-identical” sample are present in a denatured conformation.

14. (Deleted)

15. A method according to claim 1, wherein the relative total amounts of one or more compounds contained as analytes in a “nature-identical” sample, as the sum of their occurrence in phosphorylated or nonphosphorylated form and/or glycolysated and/or nonglycolysated form, are determined.

16. A method according to claim 1, wherein the relative amounts of one or more compounds contained as analytes in a “nature-identical” sample, in each case of their occurrence in phosphorylated and/or nonphosphorylated form and/or glycolysated and/or nonglycolysated form, are determined for one or more of said forms.

17. A method according to claim 1, wherein the degree of activation of one or more analytes contained in a “nature-identical” sample is determined.

18. A method according to claim 1, wherein the degree of phosphorylation and/or the degree of glycolysation of one or more analytes contained in a “nature-identical” sample is determined.

19. (Deleted)

20. A method according to claim 1, wherein said “nature-identical” sample and one or more comparison samples are taken from the same source of origin at different times, and that temporal changes of the relative amounts of one or more compounds in phosphorylated and/or nonphosphorylated form and/or glycolysated and/or nonglycolysated form contained as analytes in these samples are determined.

21. A method according to claim 1, wherein one or more of said samples are dissolved and/or diluted in a liquid dilution medium, before their deposition on said evanescent field sensor platform as a solid support, and wherein different dilutions of a sample are then deposited in different discrete measurement areas on said evanescent field sensor platform.

22. (Deleted)

23. (Deleted)

24. A method according to claim 1, wherein different samples are taken from the same organism or the same cell culture.

25. A method according to claim 24, wherein different samples are taken from different positions on the same organism.

26. A method according to claim 1, wherein different samples are taken from different organisms or different cell cultures.

27. A method according to claim 1, wherein one or more samples are mixed with a solution of polymers or polymerizable monomers, optionally in the presence of initiators or of chemical cross-linkers (e.g. glutaraldehyde), prior to their deposition on the evanescent field sensor platform as a solid support (in order to improve their adhesion on said solid support and to improve the homogeneity of the deposition).

28. A method according to claim 27, wherein said solution of polymers, polymerizable monomers or chemical cross-linkers is selected from the group comprising solutions of polysaccharides, such as agarose, or of acrylamides, or of glutaralehyde etc.

29. A method according to claim 27, wherein the mixture of the one or more samples with a solution of polymers or polymerizable monomers, optionally in the presence of initiators or of chemical cross-linkers (e.g. glutaraldehyde), leads to immobilization of a three-dimensional network structure on the evanescent field sensor platform as a solid substrate, with sample components embedded therein, which are accessible for tracer reagents in the consecutive step of a bioaffinity reaction.

30. A method according to claim 1, wherein the samples are deposited laterally selectively in discrete measurement areas, directly on the evanescent field sensor platform or on an adhesion-promoting layer deposited thereon, by means of a method selected from the group of methods comprising ink jet spotting, mechanical spotting by pen, pin or capillary, “micro contact printing”, fluidic contacting of the measurement areas with the samples through their supply in parallel or crossed micro channels, with application of pressure differences or electrical or electromagnetic potentials, and photochemical or photolithographic immobilization methods.

31. A method according to claim 1, wherein regions between the discrete measurement areas are “passivated” in order to minimize nonspecific binding of tracer compounds, i.e. that compounds which are “chemically neutral” (i.e. nonbinding) towards the analytes and other contents of the deposited samples and towards the tracer compounds for said analytes are deposited between the laterally separated measurement areas.

32. (Deleted)

33. A method according to claim 1, wherein the analytes which are to be determined and are contained in the samples deposited in discrete measurement areas are compounds of the group comprising proteins, such as monoclonal or polyclonal antibodies and antibody fragments, peptides, enzymes, glycoueptides, oligosaccharides, lectins, antigens for antibodies, proteins functionalized with additional binding sites (“tag proteins”, such as “histidine tag proteins”) and nucleic acids (e.g. DNA, RNA).

34. A method according to claim 33, wherein the analytes which are to be determined and are contained in the samples deposited in discrete measurement areas are compounds of the group comprising cytosolic or membrane-bound cell proteins, especially proteins involved in the processes of signal transduction in cells, such as kinases.

35. A method according to claim 1, wherein, as a consequence of the binding of tracer compounds to analytes which are contained in the samples in discrete measurement areas, the changes in opto-electronic signals which to be determined in a laterally resolved manner are caused by local changes in the resonance conditions for the generation of a surface plasmon in a thin metal layer as part of said evanescent field sensor platform.

36. (Deleted)

37. (Deleted)

38. A method according to claim 1, wherein, as a consequence of the binding of tracer compounds to analytes which are contained in the samples in discrete measurement areas, the changes in opto-electronic signals which to be determined in a laterally resolved manner are caused by local changes in the effective refractive index in these regions on said evanescent field sensor platform.

39. A method according to claim 1, wherein, as a consequence of the binding of tracer compounds to analytes which are contained in the samples in discrete measurement areas, the changes in opto-electronic signals which to be determined in a laterally resolved manner are caused by local changes in one or more luminescences from molecules capable of luminescence, which are located within the evanescent field of said evanescent field sensor platform.

40. A method according to claim 39, wherein said changes in one or more luminescences originate from molecules or nanoparticles capable of luminescence, which are bound as luminescence labels to one or more tracer compounds for the analytes contained in discrete measurement areas.

41. A method according to claim 40, wherein two or more luminescence labels with different emission wavelengths and/or different excitation spectra, preferably with different emission wavelengths and identical excitation wavelength, are applied for analyte detection.

42. A method according to claim 40, wherein two or more luminescence labels with different emission decay times are applied for analyte detection.

43. A method according to claim 41, wherein two or more luminescence labels are applied for the detection of different analytes in a sample.

44. A method according to claim 41, wherein two or more luminescence labels are applied for the detection of different analytes in a measurement area.

45. A method according to claim 39, wherein the excitation light is irradiated in pulses with a duration between 1 fs and 10 minutes and the emission light from the measurement areas is measured in a time-resolved manner.

46. A method according to claim 38, wherein the evanescent field sensor platform, as a solid substrate, comprises an optical waveguide, comprising one or more layers.

47. A method according to claim 46, wherein the evanescent field sensor platform as solid substrate comprises a planar optical waveguide, comprising( one or more layers, this waveguide being continuous or partitioned in discrete waveguiding regions.

48. A method according to claim 47, wherein the evanescent field sensor platform as a solid substrate comprises a planar optical thin-film waveguide with an essentially optically transparent waveguiding layer (a) on a second, likewise essentially optically transparent layer (b) with lower refractive index than layer (a) and optionally with a likewise essentially optically transparent intermediate layer (b) between layers (a) and (b), with likewise lower refractive index than layer (a).

49. A method according to claim 1, wherein excitation light from one or more light sources is in-coupled into a waveguiding layer of an evanescent field sensor platform using one or more optical in-coupling elements from the group comprising prism couplers, evanescent couplers comprising, joined optical waveguides with overlapping evanescent fields, front face (butt) couplers with focusing lenses, preferably cylindrical lenses, arranged in front of a front face (distal end) of the waveguiding layer, and grating couplers.

50. A method according to claim 49, wherein the in-coupling, of excitation light into a waveguiding layer of the evanescent field sensor platform is performed using one or more grating structures (c) that are formed in said waveguiding layer.

51. A method according to claim 1, wherein the out-coupling of light guided in a waveguiding, layer of an evanescent field sensor platform is performed using one or more grating structures (c) which are formed in said waveguiding layer and have similar or different grating period and grating depth as (,rating structures (c).

52. A method according to claim 50, wherein excitation light from one or more light sources is in-coupled into a waveguiding layer of said evanescent field sensor platform using one or more grating structures (c), directed as a guided wave towards measurement areas located on the evanescent field sensor platform, wherein furtheron luminescence from molecules capable of luminescence, which is generated in the evanescent field of said guided wave, is measured in a locally resolved manner using one or more detectors, and wherein the relative concentration of one or more analytes is determined from the relative intensity of these luminescence signals.

53. A method according to claim 39, wherein changes of the effective refractive index on the measurement areas are determined in addition to the determination of one or more luminescences.

54. A method according to claim 35, wherein determinations of the one or more luminescences and/or determinations of light signals at the excitation wavelength are performed as polarization-selective measurements.

55. A method according to claim 39, wherein the one or more luminescences are measured at a polarization that is different from the polarization of the excitation light.

56. An analytical platform for the analysis of of multiple “mature-identical” samples for analytes contained therein, being of biological relevance as binding partners in bioaffinity reactions, comprising
an evanescent field sensor platform as a solid substrate
at least one one- or two-dimensional array of discrete measurement areas with binding partners for the determination of said analytes in a bioaffinity reaction, immobilized in said measurement areas on the evanescent field sensor platform,
wherein said discrete measurement areas are generated by deposition of said “nature-identical” samples or of dilutions derived therefrom, of the same relative molecular composition as the original samples, containing the analytes to be determined as a first plurality of specific binding partners, and wherein the one or more immobilized binding partners forming the first plurality of specific binding partners are the one or more analytes themselves contained in said “nature-identical” samples.

57. An analytical platform according to claim 56, wherein the relative molecular composition of a first plurality of specific binding partners as analytes immobilized in a measurement area is identical to the original relative molecular composition of the sample applied to said measurement area.

58. An analytical platform according to claim 56, wherein the evanescent field sensor platform comprises an adhesion-promoting layer, on which the samples or their dilutions are deposited, for an improvement of the adhesion of the “nature-identical” samples or of their dilutions deposited in discrete measurement areas.

59. An analytical platform according to claim 58, wherein the adhesion-promoting layer has a thickness of less than 200 nm, preferably less than 20 nm.

60. An analytical platform according to claim 58, wherein said adhesion-promiioting layer comprises compounds of the group of silanes, functionalized silanes, epoxides, functionalized, charged or polar polymers and “self-organized passive or functionalized mono- or multi-layers”, thiols, alkyl phosphates and alkyl phosphonates, multi-functional block copolymers, Such as poly(L)lysin/polyethylene glycols.

61. (Deleted)

62. An analytical platform according to claim 56, wherein said “nature-identical” samples are selected from the group consisting of extracts of stimulated (treated) or untreated cells and extracts of healthy or diseased tissue, extracts of healthy or diseased cells (for example of human, animal, bacterial or plant cell extracts), extracts of human or animal tissue, such as organ, skin, hair or bone tissue, or of plant tissue, and comprising( body fluids or their constituents, such as blood, serum or plasm, synovial liquids, lacrimal fluid, urine, saliva, tissue fluid, lymph.

63. (Deleted)

64. An analytical platform according to claim 56, wherein said “nature-identical” samples have been taken from an organism or tissue or cellular assembly or cell by means of a method of the group of tissue slicing, biopsy and laser capture micro dissection.

65. (Deleted)

66. An analytical platform according to claim 56, wherein a deposited sample comprises the material of less than 1000 cells.

67. An analytical platform according to claim 56, wherein analytes, i.e. especially biopolymers such as nucleic acids or proteins, contained in a “nature-identical” sample are present in a native conformation.

68. An analytical platform according to claim 56, wherein analytes, i.e. especially biopolymers such as nucleic acids or proteins, contained in a “nature-identical” sample are present in a denatured conformation.

69. (Deleted)

70. An analytical platform according to claim 56, wherein one or more of said “natural-identical” samples have been dissolved and/or diluted in a liquid dilution medium, before their deposition on said evanescent field sensor platform as a solid support, and wherein different dilutions of a sample have been deposited in different discrete measurement areas on said evanescent field sensor platform.

71. (Deleted)

72. (Deleted)

73. An analytical platform according to claim 56, wherein different deposited samples have been taken from the same organism or the same cell culture.

74. An analytical platform according to claim 73, wherein different deposited samples have been taken from different positions on the same organism.

75. An analytical platform according to claim 56, wherein different deposited samples have been taken from different organisms or from different cell cultures.

76. An analytical platform according to claim 56, wherein one or more samples are mixed with a solution of polymers or polymerizable monomers, optionally in the presence of initiators or of chemical cross-linkers (e.g. glutaraldehyde), prior to their deposition on the evanescent field sensor platform as a solid support (in order to improve their adhesion on said solid Support and to improve the homogeneity of the deposition).

77. An analytical platform according to claim 76, wherein said solution of polymers, polymerizable monomers or chemical cross-linkers is selected from the group comprising solutions of polysaccharides, such as agarose, or of acrylamides, or of glutaralehyde etc.

78. An analytical platform according to claim 76, wherein the mixture of the one or more samples with a solution of polymers or polymerizable monomers, optionally in the presence of initiators or of chemical cross-linkers (e.g. glutaraldehyde), leads to immobilization of a three-dimensional network structure on the evanescent field sensor platform as a solid substrate, with sample components embedded therein, which are accessible for tracer reagents in the consecutive step of a bioaffinity reaction.

79. An analytical platform according to claim 56, wherein an array comprises more than 50, preferably more than 500, most preferably more than 5000 measurement areas.

80. An analytical platform according to claim 56, wherein the measurement areas of an array are arranged in a density of more than 10, preferably more than 100, most preferably more than 1000 measurement areas per square centimeter.

81. An analytical platform according to claim 56, wherein multiple arrays of measurement areas are provided on an evanescent field sensor platform as a solid support.

82. An analytical platform according to claim 81, wherein at least 5, preferably at least 50 arrays of measurement areas are provided on an evanescent field sensor platform as a solid support.

83. An analytical platform according to claim 56, wherein regions between the discrete measurement areas are “passivated” in order to minimize nonspecific binding of tracer compounds, i.e., that compounds, which are “chemically neutral” (i.e., nonbinding) towards the analytes and the other contents of the deposited samples and the tracer compounds for said analytes, are deposited between the laterally separated measurement areas.

84. (Deleted)

85. An analytical platform according to claim 56, wherein the analytes which are to be determined and are contained in the samples deposited in discrete measurement areas are compounds of the group comprising proteins, such as monoclonal or polyclonal antibodies and antibody fragments, peptides, enzymes, glycopeptides, oligosaccharides, lectins, antigens for antibodies, proteins functionalized with additional binding sites (“tag proteins”, such as “histidine tag proteins”) and nucleic acids (e.g. DNA, RNA).

86. An analytical platform according to claim 56, wherein the analytes which are to be determined and are contained in the samples deposited in discrete measurement areas are compounds of the group comprising cytosolic or membrane-bound cell proteins, especially proteins involved in the processes of signal transduction in cells, such as kinases.

87. An analytical platform according to claim 56, wherein the evanescent field sensor platform comprises a thin metal layer, optionally on an intermediate layer with refractive index preferably <1.5, such as silicon dioxide or magnesium fluoride, located beneath, and wherein the thickness of the metal layer and of the optional intermediate layer is selected in such a way that a surface plasmon can be excited at the wavelength of an irradiated excitation light and/or of a generated luminescence.

88. An analytical platform according to claim 87, wherein the metal is selected from the group comprising gold and silver.

89. (Deleted)

90. An analytical platform according to claim 56, wherein the evanescent field sensor platform, as a solid substrate, comprises an optical waveguide, comprising one or more layers.

91. An analytical platform according to claim 90, wherein the evanescent field sensor platform as solid substrate comprises a planar optical waveguide, comprising one or more layers, this waveguide being continuous or partitioned in discrete waveguiding regions.

92. An analytical platform according to claim 91, wherein the evanescent field sensor platform as a solid substrate comprises a planar optical thin-film waveguide with an essentially optically transparent waveguiding layer (a) on a second, likewise essentially optically transparent layer (b) with lower refractive index than layer (a) and optionally with a likewise essentially optically transparent intermediate layer (b) between layers (a) and (b), with likewise lower refractive index than layer (a).

93. An analytical platform according to claim 56, wherein a waveguiding layer of the evanescent field sensor platform is in optical contact with one or more optical coupling elements enabling the in-coupling of excitation light from one or more light sources into said waveguiding layer, said optical coupling elements being selected from the group comprising prism couplers, evanescent couplers comprising joined optical waveguides with overlapping evanescent fields, front face (butt) couplers with focusing lenses, preferably cylindrical lenses, arranged in front of a front face (distal end) of the waveguiding layer, and grating couplers.

94. An analytical platform according to claim 93, wherein one or more grating structures (c) are provided in a waveguiding layer of the evanescent field sensor platform, allowing the in-coupling, of excitation light from one or more light sources.

95. An analytical platform according to claim 56, wherein grating structures (c′), with similar or different grating period and grating depth as grating structures (c) are provided in a waveguiding layer of the evanescent field sensor platform, allowing the out-coupling of light guided in said waveguiding layer.

96. The use of a method according claim 1 for quantitative and/or qualitative analyses for the determination of chemical, biochemical or biological analytes in screening methods in pharmaceutical research, combinatorial chemistry, clinical and pre-clinical development, for real-time binding studies and the determination of kinetic parameters in affinity screening and in research, for qualitative and quantitative analyte determinations, especially for DNA- and RNA analytics and for the determination of genomic or proteomic differences in the genome, such as single nucleotide polymorphisms, for the measurement of protein-DNA interactions, for the determination of control mechanisms for mRNA expression and for the protein (bio)synthesis, for the generation of toxicity studies and the determination of expression profiles, especially for the determination of biological and chemical marker compounds, such as mRNA, proteins, peptides or small-molecular organic (messenger) compounds, and for the determination of antibodies, antigens, pathogens or bacteria in pharmaceutical product research and development, human and veterinary diagnostics, agrochemical product research and development, for symptomatic and pre-symptomatic plant diagnostics, for patient stratification in pharmaceutical product development and for the therapeutic drug selection, for the determination of pathogens, nocuous agents and germs, especially of salmonella, prions, viruses and bacteria, especially in food and environmental analytics.

97. The use of an analytical platform according to claim 56 for quantitative and/or qualitative analyses for the determination of chemical, biochemical or biological analytes in screening methods in pharmaceutical research, combinatorial chemistry, clinical and pre-clinical development, for real-time binding studies and the determination of kinetic parameters in affinity screening, and in research, for qualitative and quantitative analyte determinations, especially for DNA- and RNA analytics and for the determination of genomic or proteomic differences in the genome, such as single nucleotide polymorphisms, for the measurement of protein-DNA interactions, for the determination of control mechanisms for mRNA expression and for the protein (bio)synthesis, for the generation of toxicity studies and the determination of expression profiles, especially for the determination of biological and chemical marker compounds, such as mRNA, proteins, peptides or small-molecular organic (messenger) compounds, and for the determination of antibodies, antigens, pathogens or bacteria in pharmaceutical product research and development, human and veterinary diagnostics, agrochemical product research and development, for symptomatic and pre-symptomatic plant diagnostics, for patient stratification in pharmaceutical product development and for the therapeutic drug selection, for the determination of pathogens, nocuous a(gents and germs, especially of salmonella, prions, viruses and bacteria, especially in food and environmental analytics.

98. The use of a method according to claim 1 and an analytical platform comprising
an evanescent field sensor platform as a solid substrate
at least one one- or two-dimensional array of discrete measurement areas with binding partners for the determination of said analytes in a bioaffinity reaction, immobilized in said measurement areas on the evanescent field sensor platform,
wherein said discrete measurement areas are generated by deposition of said “nature-identical” samples or of dilutions derived therefrom, of the same relative molecular composition as the original samples, containing the analytes to be determined as a first plurality of specific binding partners, and wherein the one or more immobilized binding partners forming the first plurality of specific binding partners are the one or more analytes themselves contained in said “nature-identical” samples,
for quantitative and/or qualitative analyses for the determination of chemical, biochemical or biological analytes in screening methods in pharmaceutical research, combinatorial chemistry, clinical and pre-clinical development, for real-time binding studies and the determination of kinetic parameters in affinity screening and in research, for qualitative and quantitative analyte determinations, especially for DNA- and RNA analytics and for the determination of genomic or proteomic differences in the genome, such as single nucleotide polymorphisms, for the measurement of protein-DNA interactions, for the determination of control mechanisms for mRNA expression and for the protein (bio)synthesis, for the generation of toxicity studies and the determination of expression profiles, especially for the determination of biological and chemical marker compounds, such as mRNA, proteins, peptides or small-molecular organic (messenger) compounds, and for the determination of antibodies, antigens, pathogens or bacteria in pharmaceutical product research and development, human and veterinary diagnostics, agrochemical product research and development, for symptomatic and pre-symptomatic plant diagnostics, for patient stratification in pharmaceutical product development and for the therapeutic drug selection, for the determination of pathogens, nocuous agents and germs, especially of salmonella, prions, viruses and bacteria, especially in food and environmental analytics.

99. A method according to claim 39, wherein the evanescent field sensor platform, as a solid substrate, comprises an optical waveguide, comprising one or more layers.

100. A method according to claim 99, wherein the evanescent field sensor platform as solid substrate comprises a planar optical waveguide, comprising one or more layers, this waveguide being continuous or partitioned in discrete waveguiding regions.

101. A method according to claim 100, wherein the evanescent field sensor platform as a solid substrate comprises a planar optical thin-film waveguide with an essentially optically transparent waveguiding layer (a) on a second, likewise essentially optically transparent layer (b) with lower refractive index than layer (a) and optionally with a likewise essentially optically transparent intermediate layer (b′) between layers (a) and (b), with likewise lower refractive index than layer (a).

102. A method according to claim 38, wherein excitation light from one or more light sources is in-coupled into a waveguiding layer of an evanescent field sensor platform using one or more optical in-coupling elements from the group comprising prism couplers, evanescent couplers comprising joined optical waveguides within overlapping evanescent fields, front face (butt) couplers with focusing lenses, preferably cylindrical lenses, arranged in front of a front face (distal end) of the waveguiding layer, and grating couplers.

103. A method according to claim 102, wherein the in-coupling of excitation light into a waveguiding layer of the evanescent field sensor platform is performed using one or more grating structures (c) that are formed in said waveguiding layer.

104. A method according to claim 39, wherein excitation light from one or more light sources is in-coupled into a waveguiding layer of an evanescent field sensor platform using one or more optical in-coupling elements from the group comprising prism couplers, evanescent couplers comprising joined optical waveguides with overlapping evanescent fields, front face (butt) couplers with focusing lenses, preferably cylindrical lenses, arranged in front of a front face (distal end) of the waveguiding layer, and grating couplers.

105. A method according to claim 104, wherein the in-coupling of excitation light into a waveguiding layer of the evanescent field sensor platform is performed using one or more grating structures (c) that are formed in said waveguiding layer.
